(19) United States
(12) Patent Application Publication
(10) Pub. No.: US 2013/0309229 A1
(43) Pub. Date: Nov. 21, 2013

(54) RECOMBINANT T CELL LIGANDS AND ANTIBODIES THAT BIND B CELLS FOR THE TREATMENT OF AUTOIMMUNE DISEASES

(71) Applicants: Jianhua Yang, Palo Alto, CA (US); Jianya Huan, Beaverton, OR (US); Arthur A. Vandenbark, Portland, OR (US)

(72) Inventors: Jianhua Yang, Palo Alto, CA (US); Jianya Huan, Beaverton, OR (US); Arthur A. Vandenbark, Portland, OR (US)

(73) Assignees: THE UNITED STATES GOVERNMENT AS REPRESENTED BY THE DEPARTMENT OF VETERANS AFFAIRS, WASHINGTON, DC (US); OREGON HEALTH & SCIENCE UNIVERSITY, Portland, OR (US)

(21) Appl. No.: 13/981,888

(22) PCT Filed: Jan. 26, 2012
(86) PCT No.: PCT/US12/22770

§ 371 Date: Jul. 25, 2013

Related U.S. Application Data
(60) Provisional application No. 61/437,316, filed on Jan. 28, 2011.

(51) Int. Cl. A61K 39/00; A61K 45/06; A61K 39/395

(52) U.S. Cl. CPC A61K 39/0008; A61K 39/3955; A61K 45/06
USPC 424/133.1; 424/173.1

(57) ABSTRACT

Methods are disclosed for treating or inhibiting an autoimmune disease in a subject. In some embodiments, the disclosed methods include administering to the subject a therapeutically effective amount of one or more Major Histocompatibility Complex (MHC) molecules including covalently linked first, second and third domains; wherein the first domain is an MHC class II β1 domain and the second domain is an MHC class II α1 domain, wherein the amino terminus of the α1 domain is covalently linked to the carboxy terminus of the β1 domain; or wherein the first domain is an MHC class I α1 domain and the second domain is an MHC class I α2 domain, wherein the amino terminus of the α2 domain is covalently linked to the carboxy terminus of the α1 domain; and wherein the third domain is covalently linked to the first domain and comprises an antigen associated with the autoimmune disorder. The method also includes administering a therapeutically effective amount of one or more antibodies that bind to B cells, for example an antibody that specifically binds CD20. In specific non-limiting examples, the autoimmune disease is multiple sclerosis or rheumatoid arthritis.

### CROSS REFERENCE TO RELATED APPLICATION

This claims the benefit of U.S. Provisional Application No. 61/437,316, filed Jan. 28, 2011, which is incorporated herein by reference in its entirety.

### STATEMENT OF GOVERNMENT SUPPORT

This invention was made with United States government support pursuant to Grant No. R41MD001833 from the National Institutes of Health. The United States government has certain rights in the invention.

### FIELD

The present disclosure relates to methods for treating autoimmune disease, particularly the treatment of rheumatoid arthritis or multiple sclerosis.

### BACKGROUND

Arthritis, an inflammatory disease that affects the synovial membranes of one or more joints in the body, is the most common type of joint disease. Billions of dollars are spent annually for the treatment of arthritis and for lost days of work associated with the disease. The disease is usually oligoarticular (affects few joints), but may be generalized. The joints commonly involved include the hips, knees, lower lumbar and cervical vertebrae, proximal and distal interphalangeal joints of the fingers, first carpometacarpal joints, and first tarsometatarsal joints of the feet.

One type of arthritis is reactive arthritis, which is an acute nonpurulent arthritis secondary to a urinary tract or gastrointestinal infection with a variety of microorganisms, including Chlamydia trachomatis, Yersinia, Salmonella, Shigella, and Campylobacter. Microbial components (and not live organisms) are found in the affected joints. The arthritis appears abruptly and tends to involve the knees and ankles, but sometimes involves the wrists, fingers, and/or toes. Untreated, the arthritis lasts for about a year, then generally abates and only rarely is accompanied by ankylosing spondylitis. Despite evidence of disease being triggered by bacterial infection, viable bacteria are rarely present in affected joints and antibiotic treatment seldom provides relief.

Rheumatoid Arthritis (RA) is a chronic, systemic, inflammatory disease that affects the synovial membranes of multiple joints. RA is considered an acquired autoimmune disease, and genetic factors appear to play a role in its development. In most cases of RA, the subject has remissions and exacerbations of the symptoms. Rarely does the disease resolve completely, although at times the symptoms might temporarily remit.

Symptomatic medications, such as non-steroidal anti-inflammatory agents and aspirin, analgesics, and glucocorticoids, are used in the treatment of RA to help reduce joint pain, stiffness and swelling. In addition, low doses of methotrexate, leflunomide, D-penicillamine, sulfasalazine, gold therapy, minocycline, azathioprine, hydroxychloroquine (and other anti-malarials), and cyclosporine are used to modify the progression of the disease. In some cases, RA is also treated with monoclonal antibodies (such as a monoclonal antibody that specifically binds to CD20) or a biologic modifier. However, a need still remains for other agents that can be used to alter the progression or ameliorate the symptoms of this disease.

### SUMMARY

Methods are disclosed for treating or inhibiting an autoimmune disease in a subject. In some embodiments, the disclosed methods include administering to the subject a therapeutically effective amount of one or more Major Histocompatibility Complex (MHC) molecules including covalently linked first, second and third domains; wherein the first domain is an MHC class II β1 domain and the second domain is an MHC class II α1 domain, wherein the amino terminus of the α1 domain is covalently linked to the carboxy terminus of the β1 domain; or wherein the first domain is an MHC class I α1 domain and the second domain is an MHC class I α2 domain, wherein the amino terminus of the α2 domain is covalently linked to the carboxy terminus of the α1 domain; and wherein the third domain is covalently linked to the first domain and comprises an antigen associated with the autoimmune disorder. In other examples, the methods include administering to the selected subject a therapeutically effective amount of an MHC molecule including covalently linked first and second domains, wherein the first domain is an MHC class II β1 domain and the second domain is an MHC class II α1 domain, wherein the amino terminus of the α1 domain is covalently linked to the carboxy terminus of the β1 domain; or wherein the first domain is an MHC class I α1 domain and the second domain is an MHC class I α2 domain, wherein the amino terminus of the α2 domain is covalently linked to the carboxy terminus of the α1 domain. In some examples, the MHC molecule does not include an MHC class II α2 domain or an MHC class II β2 domain. In other examples, the MHC molecule does not include an MHC class I α3 domain. The methods also include administering a therapeutically effective amount of one or more antibodies that bind to B cells. In specific non-limiting examples, the autoimmune disease is multiple sclerosis or rheumatoid arthritis.

In some embodiments, the antibody that binds to B cells is an antibody that specifically binds to CD20, CD22, CD19, CD40, CD80, or B-lymphocyte stimulator (BLyS). In a particular example, the antibody specifically binds to CD20 (such as a monoclonal antibody that specifically binds to CD20). The monoclonal antibody that specifically binds CD20 can be a chimeric, humanized, or fully human monoclonal antibody. In one specific non-limiting example, the monoclonal antibody is rituximab.

The MHC molecule can include different antigens, which are selected based on the disorder to be treated. In some embodiments, the antigen is glycosylated or citrullinated. In some embodiments, the autoimmune disease is rheumatoid arthritis, and the antigen is collagen type II, fibrinogen-α, vimentin, α-enolase, or cartilage glycoprotein 39. In other embodiments the autoimmune disease is multiple sclerosis, and the antigen is a myelin protein, such as myelin oligodendrocyte glycoprotein (MOG), myelin basic protein (MBP), or proteolipid protein (PLP). The foregoing and other features of the disclosure will become more apparent from the following detailed description, which proceeds with reference to the accompanying figures.

### BRIEF DESCRIPTION OF THE FIGURES

FIG. 1A-C shows the nucleotide and amino acid sequence of human HLA-DR4-derived RTL362 (SEQ ID NOs: 1 and 2; FIG. 1A), and the antigen and linker included in RTL363 (SEQ ID NOs: 3 and 4; FIG. 1B) and RTL363GI (SEQ ID NOs: 5 and 6; FIG. 1C).

FIG. 2A-C is a series of diagrams showing predicted structure of MHC class II polypeptides. FIG. 2A is a model of an HLA-DR2 polypeptide on the surface of an antigen presenting cell (APC). FIG. 2B is a model of an exemplary MHC class II β1α1 molecule. FIG. 2C is a model of an exemplary β-sheet platform from a HLA-DR2 β1α1 molecule showing the hydrophobic residues.

FIG. 3A-C shows the nucleotide and amino acid sequence of murine I-A^{q}-derived RTL2000 (SEQ ID NOs: 7 and 8; FIG. 3A) and the antigen and linker included in RTL2001 (SEQ ID NOs: 9 and 10; FIG. 3B) and RTL2001MII (SEQ ID NOs: 11 and 12; FIG. 3C).

FIG. 4 is a graph showing the mean disease score in a study of mice with collagen-induced arthritis. At day 76 post-immunization, four mice from each of the control (RTL2000) and RTL2001MII pre-treated groups were selected. Two mice from each group were treated with 250 μg rituximab (human anti-CD20 antibody) on days 76 and 78 (rituximab in Ctrl group and rituximab in RTL2001MII group). Two mice from each group were treated with a single dose of 250 μg mouse anti-CD20 antibody on day 76 (anti-HIM CD20 mAb in Ctrl group and anti-H/M CD20 mAb in RTL2001MII group).

FIG. 5 is a bar graph showing the disease score in the mice shown in FIG. 3 at day 95 as a percentage of the disease score at day 76.

FIG. 6 is a graph showing the incidence of collagen-induced arthritis in transgenic DR4 mice treated with vehicle (control), RTL362 (empty RTL), or RTL363 (RTL362 with human collagen II 261-273 peptide).

### SEQUENCE LISTING

The nucleic acid and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NOs: 1 and 2 are the nucleic acid and amino acid sequences, respectively, of an exemplary MHC class II β1α1 molecule derived from human HLA-DR4 (RTL362).

SEQ ID NOs: 3 and 4 are the nucleic acid and amino acid sequences, respectively, of a human collagen II 261-273 peptide and linker sequence.

SEQ ID NOs: 5 and 6 are the nucleic acid and amino acid sequences, respectively, of a human collagen II 259-273 peptide and linker.

SEQ ID NOs: 7 and 8 are the nucleic acid and amino acid sequences, respectively, of an exemplary MHC class II β1α1 molecule derived from mouse I-A^{q} (RTL2000).

SEQ ID NOs: 9 and 10 are the nucleic acid and amino acid sequences, respectively, of a bovine collagen II 257-270 peptide and linker.

SEQ ID NOs: 11 and 12 are the nucleic acid and amino acid sequences, respectively, of a modified bovine collagen II 257-270 peptide and linker.

SEQ ID NO: 13 is the amino acid sequence of an exemplary human CD20 molecule.

SEQ ID NO: 14 is the amino acid sequence of an exemplary human MHC class II β1α1 molecule.

SEQ ID NO: 15 is the amino acid sequence of a MOG 35-55 peptide.

SEQ ID NO: 16 is the amino acid sequence of a MOG 1-25 peptide.

SEQ ID NO: 17 is the amino acid sequence of a MOG 94-116 peptide.

SEQ ID NO: 18 is the amino acid sequence of a MOG 145-160 peptide

SEQ ID NO: 19 is the amino acid sequence of a MOG 194-208 peptide.

SEQ ID NO: 20 is the amino acid sequence of an MBP 10-30 peptide.

SEQ ID NO: 21 is the amino acid sequence of an MBP 35-45 peptide.

SEQ ID NO: 22 is the amino acid sequence of an MBP 77-91 peptide.

SEQ ID NO: 23 is the amino acid sequence of an MBP 85-99 peptide.

SEQ ID NO: 24 is the amino acid sequence of an MBP 95-112 peptide.

SEQ ID NO: 25 is the amino acid sequence of an MBP 145-164 peptide.

SEQ ID NO: 26 is the amino acid sequence of a PLP 139-151 peptide.

SEQ ID NO: 27 is the amino acid sequence of a PLP 95-116 peptide.

SEQ ID NO: 28 is the amino acid sequence of a collagen II 261-274 peptide.

SEQ ID NO: 29 is the amino acid sequence of a collagen II 259-273 peptide.

SEQ ID NO: 30 is the amino acid sequence of a collagen II 257-270 peptide.

SEQ ID NO: 31 is the amino acid sequence of a modified collagen II 257-270 peptide.

SEQ ID NO: 32 is the amino acid sequence of a fibrinogen-α 40-59 peptide.

SEQ ID NO: 33 is the amino acid sequence of a fibrinogen-α 616-625 peptide.

SEQ ID NO: 34 is the amino acid sequence of a fibrinogen-α 79-91 peptide.

SEQ ID NO: 35 is the amino acid sequence of a fibrinogen-α 121-140 peptide.

SEQ ID NO: 36 is the amino acid sequence of a vimentin 59-79 peptide.

SEQ ID NO: 37 is the amino acid sequence of a vimentin 26-44 peptide.

SEQ ID NO: 38 is the amino acid sequence of a vimentin 256-275 peptide.

SEQ ID NO: 39 is the amino acid sequence of a vimentin 415-433 peptide.

SEQ ID NO: 40 is the amino acid sequence of an α-enolase 5-21 peptide.

SEQ ID NO: 41 is the amino acid sequence of a cartilage glycoprotein 259-271 peptide.

SEQ ID NO: 42 is the amino acid sequence of an exemplary mouse MHC class II β1α1 molecule.

SEQ ID NOs: 43-45 are the amino acid sequences of exemplary linker peptides.

### DETAILED DESCRIPTION

Anti-CD20 antibodies (such as rituximab) are an effective treatment option for RA. However, only about 30% of RA patients respond to rituximab treatment. Furthermore, in order to minimize potential side effects, there is currently a nine-month waiting period following initial rituximab treatment before another RA treatment can be used. The MHC molecules (recombinant T Cell Ligands; RTLs) disclosed herein are candidates for combination treatment with anti-CD20 antibody that can improve the efficacy of antibody treatment alone.

It has been demonstrated that anti-CD20 antibodies can be used to treat autoimmune disease, such as multiple sclerosis and rheumatoid arthritis. Additional antibodies that bind B cells (for example, specifically binds CD22, CD19, CD40, CD80, or BLyS) can also be used to treat autoimmune disease. However, a need remains for methods to treat autoimmune diseases, including combinations of agents that can be used to provide a synergistic effect, in order to provide superior efficacy and reduced side effects. Disclosed herein are methods that utilize a therapeutically effective amount of an RTL in combination with a therapeutically effective amount of an antibody that binds B cells (for example, specifically binds CD20, CD22, CD19, CD40, CD80, or BLyS). These agents act synergistically to produce unexpectedly superior results, such as in reducing inflammation and other signs and symptoms of autoimmune disease, such as rheumatoid arthritis.

### I. ABBREVIATIONS

Ab antibody
APC antigen presenting cell
bCII bovine collagen II
BLyS B lymphocyte stimulator
CD cluster of differentiation
CIA collagen-induced arthritis
CII collagen II
mAb monoclonal antibody
MBP myelin basic protein
MHC major histocompatibility complex
MOG myelin oligodendrocyte glycoprotein
MS multiple sclerosis
PLP proteolipid protein
RA rheumatoid arthritis
RTL recombinant T cell receptor ligand

### II. TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below.

All publications, patent applications, patents, and other references mentioned herein are incorporated by reference in their entirety. All GenBank Accession numbers mentioned herein are incorporated by reference in their entirety as present in GenBank on Jan. 28, 2011. In case of conflict, the present specification, including explanations of terms, will control. In addition, the materials, methods, and examples are illustrative only and not intended to be limiting.

In order to facilitate review of the various embodiments of this disclosure, the following explanations of specific terms are provided:

α-Enolase: Also known as ENO1, enolase 1 (alpha), phosphopyruvate hydratase, and tau-crystallin (e.g., GenBank Gene ID: 2023). A homodimeric soluble enzyme; one of three enolase isoenzymes found in mammals. The α-enolase gene also encodes a shorter monomeric structural lens protein (tau-crystallin), made from the same message. The full length protein is found in the cytoplasm and the shorter protein, produced from an alternative translation start, is localized to the nucleus.

Nucleic acid and protein sequences for α-enolase are publicly available. For example, GenBank Accession No. NM_{—}001428 discloses an exemplary human α-enolase nucleic acid sequence, and GenBank Accession No. NP_{—}001419 discloses an exemplary human α-enolase protein sequence, both of which are incorporated by reference as provided by GenBank on Jan. 28, 2011. Similarly, GenBank Accession No. NM_{—}023119 discloses an exemplary mouse α-enolase nucleic acid sequence, and GenBank Accession No. NP_{—}075608 discloses an exemplary mouse α-enolase protein sequence, both of which are incorporated by reference as provided by GenBank on Jan. 28, 2011. One of skill in the art can identify additional α-enolase sequences from human, mouse, or other species.

Antibody: Immunoglobulin molecules and immunologically active portions of immunoglobulin molecules, e.g., molecules that contain an antigen binding site that specifically binds (immunoreacts with) an antigen, such as a B cell-expressed antigen (for example, CD20, CD22, CD19, CD40, CD80, or BLyS).

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the V_{L}, V_{H}, C_{L} and C_{H1} domains; (ii) an F_{d} fragment consisting of the V_{H} and C_{H1} domains; (iii) an Fv fragment consisting of the V_{L} and V_{H} domains of a single arm of an antibody; (iv) a dAb fragment (Ward et al., Nature 341:544-546, 1989) which consists of a V_{H} domain; (v) an isolated complementarity determining region (CDR); and (vi) a F(ab′)₂ fragment, a bivalent fragment comprising two Fab fragments linked by a disulfide bridge at the hinge region.

Immunoglobulins and certain variants thereof are known and many have been prepared in recombinant cell culture (e.g., see U.S. Pat. Nos. 4,745,055 and 4,444,487; WO 88/03565; EP 256,654; EP 120,694; EP 125,023; Falkner et al., Nature 298:286, 1982; Morrison, J. Immunol. 123:793, 1979; Morrison et al., Ann. Rev. Immunol. 2:239, 1984).

Antibody fragment (fragment with specific antigen binding): Various fragments of antibodies have been defined, including Fab, (Fab′)₂, Fv, and single-chain Fv (scFv). These antibody fragments are defined as follows: (1) Fab, the fragment that contains a monovalent antigen-binding fragment of an antibody molecule produced by digestion of whole antibody with the enzyme papain to yield an intact light chain and a portion of one heavy chain or equivalently by genetic engineering; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction or equivalently by genetic engineering; (4) F(Ab′)₂, a dimer of two FAb′ fragments held together by disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Methods of making these fragments are routine in the art.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 8 amino acids (such as about 8-50 or 8-23 amino acids) in a unique spatial conformation. Methods of determining spatial conformation of epitopes include, for example, x-ray crystallography and two-dimensional nuclear magnetic resonance.

An antigen can be a tissue-specific antigen, or a disease-specific antigen. These terms are not exclusive, as a tissue-specific antigen can also be a disease-specific antigen. A tissue-specific antigen is expressed in a limited number of tissues, such as a single tissue. A tissue-specific antigen may be expressed by more than one tissue, such as, but not limited to, an antigen of the central or peripheral nervous system or an antigen of the joints. In particular examples, an antigen of the central or peripheral nervous system includes a myelin protein, such as myelin oligodendrocyte glycoprotein (MOG), myelin basic protein (MBP), proteolipid protein (PLP), or an antigenic determinant thereof. Antigens of the joints include, but are not limited to, collagen, fibrinogen-α, vimentin, α-enolase, and cartilage glycoprotein-39 or an antigenic determinant thereof. A disease-specific antigen is expressed coincidentally with a disease process. Specific non-limiting examples of a disease-specific antigen are an antigen whose expression correlates with, or is predictive of, an autoimmune disease, such as multiple sclerosis or rheumatoid arthritis. A disease-specific antigen can be an antigen recognized by T cells or B cells.

Arthritis: Arthritis is an inflammatory disease that affects the synovial membranes of one or more joints in the body. It is the most common type of joint disease, and it is characterized by the inflammation of the joint. The disease is usually oligoarticular (affects few joints), but may be generalized. The joints commonly involved include the hips, knees, lower lumbar and cervical vertebrae, proximal and distal interphalangeal joints of the fingers, first carpometacarpal joints, and first tarsometatarsal joints of the feet.

One type of arthritis is reactive arthritis, which is an acute nonpurulent arthritis secondary to a urinary tract or gastrointestinal infection with a variety of microorganisms, including Chlamydia trachomatis, Yersinia, Salmonella, Shigella, and Campylobacter. Microbial components are found in the affected joints. The arthritis appears abruptly and tends to involve the knees and ankles, but sometimes involves the wrists, fingers, and/or toes. Another type of arthritis is rheumatoid arthritis (RA). RA is a chronic, systemic, inflammatory disease that affects the synovial membranes of multiple joints in the body. Because the disease is systemic, there are many extra-articular features of the disease as well. For example, neuropathy, scleritis, lymphadenopathy, pericarditis, splenomegaly, arteritis, and rheumatoid nodules are frequent components of the disease. In most cases of RA, the subject has remissions and exacerbations of the symptoms. RA is considered an autoimmune disease that is acquired and in which genetic factors appear to play a role.

Autoimmune disorder: A disorder in which the immune system produces an immune response (for example, a B cell or a T cell response) against an endogenous antigen, with consequent injury to tissues. For example, rheumatoid arthritis is an autoimmune disorder, as are Hashimoto's thyroiditis, pernicious anemia, Addison's disease, type I diabetes, systemic lupus erythematosus, dermatomyositis, Sjogren's syndrome, dermatomyositis, lupus erythematosus, multiple sclerosis, myasthenia gravis, Reiter's syndrome, and Grave's disease, among others.

B Cell: A lymphocyte, a type of white blood cell (leukocyte), that expresses immunoglobulin on its surface and can ultimately develop into an antibody secreting plasma cell. In one example, a B cell expresses CD19. In other examples, a B cell expresses CD20, CD22, CD40, CD80, and/or BLyS. An “immature B cell” is a cell that can develop into a mature B cell. Generally, pro-B cells (that express, for example, CD45 or B220) undergo immunoglobulin heavy chain rearrangement to become pro-B pre-B cells, and further undergo immunoglobulin light chain rearrangement to become immature B cells. Immature B cells include T1 and T2 B cells. Thus, one example of an immature B cell is a T1 B that is an AA41^{hi}CD23^{lo} cell. Another example of an immature B cell is a T2 B that is an AA41^{hi}CD23^{hi} cell. Thus, immature B cells include B220 expressing cells wherein the light and the heavy chain immunoglobulin genes are rearranged, and that express AA41. Immature B cells express IgM on their cell surface and can develop into mature B cells, which can express different forms of immunoglobulin (e.g., IgA, IgG). Mature B cells may also express characteristic markers such as CD21 and CD23 (e.g. CD23^{hi}CD21^{hi} cells), but do not express AA41. B cells can be activated by agents such as lipopolysaccharide (LPS), CD40 ligation, and antibodies that crosslink the B cell receptor (immunoglobulin), including antigen, or anti-Ig antibodies.

A “regulatory B cell” (Breg) is a B cell that suppresses the immune response. Breg cells can suppress T cell activation either directly or indirectly, and may also suppress antigen presenting cells, other innate immune cells, or other B cells. Breg cells can be CD1d^{hi}CD5⁺ or express a number of other B cell markers and/or belong to other B cell subsets. These cells can also secrete IL-10. Breg cells also express TIM-1, such as TIM-1⁺CD19⁺ B cells.

B lymphocyte stimulator (BLyS): A cytokine that belongs to the tumor necrosis factor (TNF) ligand family. Also known as tumor necrosis factor (ligand) superfamily member 13b (TNFSF13B) or B cell activating factor (BAFF) (see, e.g., GenBank Gene ID No: 10673). BLyS is a 285-amino acid long peptide glycoprotein which undergoes glycosylation at residue 124. It is expressed as transmembrane protein on various cell types including monocytes, dendritic cells and bone marrow stromal cells. The transmembrane form can be cleaved from the membrane, generating a soluble protein fragment. BLyS is the natural ligand of three unusual tumor necrosis factor receptors named BAFF-R, TACI, and BCMA, all of which have differing binding affinities for it. These receptors are expressed mainly on mature B lymphocytes (TACI is also found on a subset of T-cells and BCMA on plasma cells).

Cartilage glycoprotein-39: Also known as chitinase 3-like 1 (cartilage glycoprotein-39) (e.g., GenBank Gene ID: 1116). A member of the glycoside hydrolase 18 family of chitinases. Cartilage glycoprotein-39 lacks chitinase activity and is secreted by activated macrophages, chondrocytes, neutrophils, and synovial cells and is thought to play a role in inflammation and tissue remodeling.

Nucleic acid and protein sequences for cartilage glycoprotein-39 are publicly available. For example, GenBank Accession No. NM_{—}001276 discloses an exemplary human cartilage glycoprotein-39 nucleic acid sequence, and GenBank Accession No. NP_{—}001267 discloses an exemplary human cartilage glycoprotein-39 protein sequence, both of which are incorporated by reference as provided by GenBank on Jan. 28, 2011. Similarly, GenBank Accession No. NM_{—}007695 discloses an exemplary mouse cartilage glycoprotein-39 nucleic acid sequence, and GenBank Accession No. NP_{—}031721 discloses an exemplary mouse cartilage glycoprotein-39 protein sequence, both of which are incorporated by reference as provided by GenBank on Jan. 28, 2011. One of skill in the art can identify additional cartilage glycoprotein-39 sequences from human, mouse, or other species.

CD19: Also known as B-lymphocyte surface antigen B4, T-cell surface antigen Leu-12; differentiation antigen CD19 (see, e.g., GenBank Gene ID No: 930). A marker of B cells. CD19 is a cell surface molecule which assembles with the antigen receptor of B lymphocytes in order to decrease the threshold for antigen receptor-dependent stimulation.

CD20: The CD20 protein (cluster of differentiation 20, also called human B-lymphocyte-restricted differentiation antigen or Bp35) is a hydrophobic transmembrane protein with a molecular weight of approximately 35 kD located on pre-B and mature B lymphocytes (Valentine et al., J. Biol. Chem. 264(19):11282-11287, 1989; and Einfield et al., EMBO J. 7(3):711-717, 1988). See, e.g., GenBank Gene ID No: 931. In vivo, CD20 is found on the surface of greater than 90% of B cells from peripheral blood or lymphoid organs and is expressed during early pre-B cell development and remains expressed until plasma cell differentiation. CD20 is present on both normal B cells and malignant B cells, but is not found on hematopoietic stem cells, pro-B cells, normal plasma cells, or other normal tissues (Tedder et al., J. Immunol. 135(2):973-979, 1985).

CD20 is involved in regulating early steps in the activation and differentiation process of B cells (Tedder et al., Eur. J. Immunol. 16:881-887, 1986) and can function as a calcium ion channel (Tedder et al., J. Cell. Biochem. 14D:195, 1990).

CD22: A lineage-restricted B cell antigen belonging to the immunoglobulin superfamily. See, e.g., GenBank Gene ID No: 933. CD22 is expressed in 60-70% of B cell lymphomas and leukemias. CD22 is not present on the cell surface in the early stages of B cell development or on stem cells. As used herein “CD22” refers to a CD22 polypeptide or variant or fragment thereof. Sequences of human CD22 are known in the art (see, for example Tones et al., J. Immunol. 149(8):2641-2649, 1992; and Wilson et al., J. Exp. Med. 173(1):137-146, 1991).

CD40: CD40 molecule, TNF receptor superfamily member 5 (also known as p50; Bp50 CDW40: MGC9013: TNFRSF5; CD40) (see, e.g., GenBank Gene ID No: 958). CD40 is a member of the TNF-receptor superfamily. This receptor has been found to be involved in mediating a broad variety of immune and inflammatory responses including T cell-dependent immunoglobulin class switching, memory B cell development, and germinal center formation. AT-hook transcription factor AKNA is reported to coordinately regulate the expression of this receptor and its ligand, which may be important for homotypic cell interactions. Adaptor protein TNFR2 interacts with this receptor and serves as a mediator of the signal transduction.

CD80: Also known as B7, B7-1, CD28LG (see, e.g., GenBank Gene ID No: 941). CD80 provides regulatory signals for T lymphocytes by binding to the CD28 and CTLA5 ligands of T cells.

Citrullination: Citrullination (or deimination) refers to post-translational modification of an arginine residue in a polypeptide to a citrulline residue. This reaction is catalyzed by a peptidylarginine deiminase. Citrullination of a polypeptide affects the charge of the polypeptide, as arginine is positively charged at neutral pH, while citrulline is uncharged. This may lead to changes in hydrophobicity and protein folding. In some examples, anti-citrullinated protein/peptide antibodies are detectable in subjects with an autoimmune disease (such as rheumatoid arthritis). In some examples, a citrullinated polypeptide or antigen is a polypeptide or antigen that includes at least one citrulline residue. Polypeptides that may be citrullinated include fibrinogen-α, vimentin, α-enolase, cartilage glycoprotein-39, and collagen type II, or an antigenic determinant thereof.

Collagen: The main component of connective tissue, mostly present in tendon, ligament, cartilage, skin, cornea, bone, and blood vessels. Collagen includes a large family of proteins, with at least 29 types identified (types I-XXIX). The most abundant type of collagen is type I (COL1A1 and COL1A2). Type II collagen (collagen II, COL2A1) is fibrillar collagen found in cartilage and the vitreous humor of the eye. It is a potential autoantigen in rheumatoid arthritis.

Nucleic acid and protein sequences for collagen II (e.g., GenBank Gene ID: 1280) are publicly available. For example, GenBank Accession Nos. NM_{—}001844 and NM_{—}033150 disclose exemplary human collagen II nucleic acid sequences, and GenBank Accession Nos. NP_{—}001835 and NP_{—}149162 disclose exemplary human collagen II protein sequences, each of which are incorporated by reference as provided by GenBank on Jan. 28, 2011. Similarly, GenBank Accession Nos. NM_{—}031163 and NM_{—}001113515 disclose exemplary mouse collagen H nucleic acid sequences, and GenBank Accession Nos. NP_{—}112440 and NP_{—}001106987 disclose exemplary mouse collagen II protein sequences, each of which are incorporated by reference as provided by GenBank on Jan. 28, 2011. One of skill in the art can identify additional collagen II sequences from human, mouse, or other species.

Domain: A discrete part of an amino acid sequence of a polypeptide or protein that can be equated with a particular function. For example, the α and β polypeptides that constitute a MHC class II molecule are each recognized as having two domains, α1, α2 and β1, β2, respectively. Similarly, the α chain of MHC class I molecules is recognized as having three domains, α1, α2, and α3. The various domains in each of these molecules are typically joined by linking amino acid sequences. In one embodiment, the entire domain sequence is included in a recombinant molecule by extending the sequence to include all or part of the linker or the adjacent domain. For example, when selecting the α1 domain of an MHC class II molecule, the selected sequence will generally extend from amino acid residue number 1 of the α chain, through the entire α1 domain and will include all or part of the linker sequence located at about amino acid residues 76-90 (at the carboxy terminus of the α1 domain, between the α1 and α2 domains). The precise number of amino acids in the various MHC molecule domains varies depending on the species of mammal, as well as between classes of genes within a species. The critical aspect for selection of a sequence for use in a recombinant molecule is the maintenance of the domain function rather than a precise structural definition based on the number of amino acids. One of skill in the art will appreciate that domain function may be maintained even if somewhat less than the entire amino acid sequence of the selected domain is utilized. For example, a number of amino acids at either the amino or carboxy termini of the α1 domain may be omitted without affecting domain function. Typically however, the number of amino acids omitted from either terminus of the domain sequence will be no greater than 10, and more typically no greater than 5 amino acids. The functional activity of a particular selected domain may be assessed in the context of the two-domain MHC polypeptides provided by this disclosure (e.g., the class II β1α1 or class I α1α2 polypeptides) using the antigen-specific T-cell proliferation assay as described in detail below. For example, to test a particular β1 domain, the domain will be linked to a functional a 1 domain so as to produce a β1α1 molecule and then tested in the described assay. A biologically active β1α1 or α1α2 polypeptide will inhibit antigen-specific T-cell proliferation by at least about 50%, thus indicating that the component domains are functional. Typically, such polypeptides will inhibit T-cell proliferation in this assay system by at least 75% and sometimes by greater than about 90%.

Fibrinogen-α: The alpha component of fibrinogen (e.g., GenBank Gene ID: 2243). Fibrinogen is a glycoprotein including three pairs of nonidentical polypeptide chains. Following vascular injury, fibrinogen is cleaved by thrombin to form fibrin, the major component of blood clots. In addition, various cleavage products of fibrinogen and fibrin regulate cell adhesion and spreading, have vasoconstrictor and chemotactic activity, and are mitogens. The fibrinogen-α gene encodes two isoforms which vary in the carboxy-terminus, as a result of alternative splicing.

Nucleic acid and protein sequences for fibrinogen-α are publicly available. For example, GenBank Accession Nos. NM_{—}000508 and NM_{—}021871 disclose exemplary human fibrinogen-α nucleic acid sequences, and GenBank Accession Nos. NP_{—}000499 and NP_{—}068657 disclose exemplary human fibrinogen-α protein sequences, all of which are incorporated by reference as provided by GenBank on Jan. 28, 2011. Similarly, GenBank Accession Nos. NM_{—}010196 and NM_{—}001111048 disclose exemplary mouse fibrinogen-α nucleic acid sequences, and GenBank Accession Nos. NP_{—}034326 and NP_{—}001104518 disclose exemplary mouse fibrinogen-α protein sequences, all of which are incorporated by reference as provided by GenBank on Jan. 28, 2011. One of skill in the art can identify additional fibrinogen-α sequences from human, mouse, or other species.

Glycosylation: Covalent modification of a biomolecule (such as a protein or lipid) with one or more oligosaccharide chains. Proteins having at least one oligosaccharide modification are referred to as “glycoproteins” or “glycosylated proteins.” In the case of proteins, glycosylation is usually N-linked or O-linked. N-linked glycosylation refers to linkage of an oligosaccharide to the side chain amino group of an asparagine or arginine residue in a protein. O-linked glycosylation refers to linkage of an oligosaccharide to the hydroxyl side chain of a serine, threonine, or hydroxylysine amino acid in a protein. The oligosaccharide chains of glycoproteins are enormously varied, due to the combination of various sugars (for example, N-acetylglucosamine, N-acetylgalactosamine, N-acetyllactosamine, mannose, galactose, glucose, N-acetylneuraminic acid, or fucose) and the presence of branched structures (such as biantennary, triantennary, or tetra-antennary structures).

In some examples, a glycosylated polypeptide or antigen is a polypeptide or antigen that includes at least one oligosaccharide linked to an amino acid residue of the polypeptide or antigen. In one example, a polypeptide that may be glycosylated is collagen II or an antigenic determinant thereof. In other examples, polypeptides that may be glycosylated include fibrinogen-α, vimentin, α-enolase, and cartilage glycoprotein-39, or an antigenic determinant thereof.

Immunosuppressive agent: A molecule, such as a chemical compound, small molecule, steroid, nucleic acid molecule, or other biological agent, that can decrease an immune response such as an inflammatory reaction. Immunosuppressive agents include, but are not limited to an agent of use in treating arthritis (anti-arthritis agent). Specific, non-limiting examples of immunosuppressive agents are non-steroidal anti-inflammatory agents, cyclosporin A, FK506, and anti-CD4. In additional examples, the agent is a biological response modifier, such as Kineret® (anakinra), Enbrel® (etanercept), or Remicade® (infliximab), a disease-modifying antirheumatic drug (DMARD), such as Arava® (leflunomide), a nonsteroidal anti-inflammatory drug (NSAID), specifically a cyclooxygenase-2 (COX-2) inhibitor, such as Celebrex® (celecoxib) or Vioxx® (rofecoxib), or another product, such as Hyalgan® (hyaluronan) or Synvisc® (hylan G-F20).

Immune response: A response of a cell of the immune system, such as a B cell or T cell to a stimulus. In one embodiment, the response is specific for a particular antigen (an “antigen-specific response”).

Inflammatory arthropathy: An inflammatory arthropathy is an inflammatory disease affecting one or more joints, such as an inflammatory disease that affects the synovial membranes of one or more joints. Inflammatory arthropathies include, for example, arthritis (such as RA), ankylosing spondylitis, Reiter's syndrome, psoriatic arthropathy, enteropathic spondylitis, juvenile arthropathy, and reactive arthropathy.

Isolated: An “isolated” biological component (such as a nucleic acid, peptide or protein) has been substantially separated, produced apart from, or purified away from other biological components in the cell or environment in which the component occurs, e.g., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids or peptides.

Linker: An amino acid sequence that covalently links two polypeptide domains. Linker sequences may be included in the recombinant MHC polypeptides of the present disclosure in some examples to provide rotational freedom to the linked polypeptide domains and thereby to promote proper domain folding and inter- and intra-domain bonding. By way of example, in a recombinant polypeptide comprising Ag-β1-α1 (where Ag=antigen), linker sequences may be provided between the Ag and β1 domains and/or between β1 and α1 domains. In other examples, recombinant MHC class I α1α2 polypeptides according to the present disclosure include a covalent linkage joining the carboxy terminus of the α1 domain to the amino terminus of the α2 domain. The α1 and α2 domains of native MHC class I α chains are typically covalently linked in this orientation by an amino acid linker sequence. This native linker sequence may be maintained in the recombinant constructs; alternatively, a recombinant linker sequence may be introduced between the α1 and α2 domains (either in place of or in addition to the native linker sequence).

Linker sequences, which are generally between 2 and 25 amino acids in length, are well known in the art and include, but are not limited to, the glycine(4)-serine spacer described by Chaudhary et al. (Nature 339:394-397, 1989).

MHC class I: MHC class I molecules are formed from two non-covalently associated proteins, the α chain and β2-microglobulin. The α chain comprises three distinct domains, α1, α2 and α3. The three-dimensional structure of the α1 and α2 domains forms the groove into which antigen fits for presentation to T-cells. The α3 domain is an Ig-fold like domain that contains a transmembrane sequence that anchors the α chain into the cell membrane of the APC. MHC class I complexes, when associated with antigen (and in the presence of appropriate co-stimulatory signals) stimulate CD8 cytotoxic T-cells, which function to kill any cell which they specifically recognize.

In some examples disclosed herein, an MHC class I α1α2 polypeptide includes the α1 and α2 domains of a MHC class I molecule in covalent linkage. In other examples, an α1α2 nucleic acid includes a recombinant nucleic acid sequence encoding an α1α2 polypeptide. The orientation of the polypeptide is such that the carboxy terminus of the α1 domain is covalently linked to the amino terminus of the α2 domain. An α1α2 polypeptide comprises less than the whole class I α chain, and usually omits most or all of the α3 domain of the α chain. Specific non-limiting examples of an α1α2 polypeptide are polypeptides wherein the carboxy terminus of the α1 domain is covalently linked to the amino terminus of the α2 domain of an HLA-A, HLA-B or HLA-C molecule. In one embodiment, the α3 domain is omitted from an α1α2 polypeptide, thus the α1α2 polypeptide does not include an α3 domain.

MHC Class II: MHC class II molecules are formed from two non-covalently associated proteins, the α chain and the β chain. The α chain comprises α1 and α2 domains, and the β chain comprises β1 and β2 domains. The cleft into which the antigen fits is formed by the interaction of the α1 and β1 domains. The α2 and β2 domains are transmembrane Ig-fold like domains that anchor the α and β chains into the cell membrane of the APC. MHC class II complexes, when associated with antigen (and in the presence of appropriate co-stimulatory signals) stimulate CD4 T-cells. The primary functions of CD4 T-cells are to initiate the inflammatory response, to regulate other cells in the immune system, and to provide help to B cells for antibody synthesis.

In some examples disclosed herein, an MHC class II β1α1 polypeptide includes a recombinant polypeptide comprising the α1 and β1 domains of a MHC class II molecule in covalent linkage. In other examples, a β1α1 nucleic acid includes a recombinant nucleic acid sequence encoding a β1α1 polypeptide. To ensure appropriate conformation, the orientation of the polypeptide is such that the carboxy terminus of the β1 domain is covalently linked to the amino terminus of the α1 domain. In one embodiment, the polypeptide is a human β1α1 polypeptide, and includes the α1 and β1 domains of a human MHC class II molecule. One specific, non-limiting example of a human β1α1 polypeptide is a molecule wherein the carboxy terminus of the β1 domain is covalently linked to the amino terminus of the α1 domain of an HLA-DR molecule. An additional, specific non-limiting example of a human β1α1 polypeptide is a molecule wherein the carboxy terminus of the β1 domain is covalently linked to the amino terminus of the α1 domain of an HLA-DR (either A or B), an HLA-DP (A and B), or an HLA-DQ (A and B) molecule. In one embodiment, the β1α1 polypeptide does not include a β2 domain. In another embodiment, the β1α1 polypeptide does not include an α2 domain. In yet another embodiment, the β1α1 polypeptide does not include either an α2 or a β2 domain.

Monoclonal antibody (mAb): An antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells.

Multiple sclerosis: An autoimmune disease classically described as a central nervous system white matter disorder disseminated in time and space that presents as relapsing-remitting illness in 80-85% of patients. Diagnosis can be made by brain and spinal cord magnetic resonance imaging (MRI), analysis of somatosensory evoked potentials, and analysis of cerebrospinal fluid to detect increased amounts of immunoglobulin or oligoclonal bands. MRI is a particularly sensitive diagnostic tool. MRI abnormalities indicating the presence or progression of MS include hyperintense white matter signals on T2-weighted and fluid attenuated inversion recovery images, gadolinium enhancement of active lesions, hypointensive “black holes” (representing gliosis and axonal pathology), and brain atrophy on T1-weighted studies. Serial MRI studies can be used to indicate disease progression.

Relapsing-remitting multiple sclerosis is a clinical course of MS that is characterized by clearly defined, acute attacks with full or partial recovery and no disease progression between attacks. Secondary-progressive multiple sclerosis is a clinical course of MS that initially is relapsing-remitting, and then becomes progressive at a variable rate, possibly with an occasional relapse and minor remission. Primary progressive multiple sclerosis presents initially in the progressive form.

Myelin basic protein (MBP): A myelin protein which is a major constituent of the myelin sheath of oligodendrocytes and Schwann cells in the central and peripheral nervous system, respectively. Nucleic acid and protein sequences for MBP are publicly available. For example, GenBank Accession Nos. NM_{—}001025101, NM_{—}001025100, NM_{—}001025081, NM_{—}001025090, NM_{—}001025092, and NM_{—}002385 disclose exemplary human MBP nucleic acid sequences, and GenBank Accession Nos. NP_{—}001020272, NP_{—}001020271, NP_{—}001020252, NP_{—}001020261, NP_{—}001020263, and NP_{—}002376 disclose exemplary human MBP protein sequences, all of which are incorporated by reference as provided by GenBank on Jan. 28, 2011. Similarly, GenBank Accession Nos. NM_{—}010777, NM_{—}001025245, NM_{—}001025251, NM_{—}001025254, NM_{—}001025255, NM_{—}001025256, NM_{—}001025258, and NM_{—}001025259 disclose exemplary mouse MBP nucleic acid sequences, and GenBank Accession Nos. NP_{—}034907, NP_{—}001020416, NP_{—}001020422, NP_{—}001020425, NP_{—}001020426, NP_{—}001020427, NP_{—}001020429, and NP_{—}001020430 disclose exemplary mouse MBP protein sequences, all of which are incorporated by reference as provided by GenBank on Jan. 28, 2011. One of skill in the art can identify additional MBP sequences from human, mouse, or other species.

Myelin oligodendrocyte glycoprotein (MOG): A myelin protein which is a membrane protein expressed on oligodendrocyte cell surface and the outermost surface of myelin sheaths. Nucleic acid and protein sequences for MOG are publicly available. For example, GenBank Accession Nos. NM_{—}001008228, NM_{—}001008229, NM_{—}001170418, NM_{—}002433, NM_{—}206809, NM_{—}206810, NM_{—}206811, NM_{—}206812, and NM_{—}206814 disclose exemplary human MOG nucleic acid sequences, and GenBank Accession Nos. NP_{—}001008229, NP_{—}001008230, NP_{—}001163889, NP_{—}002424, NP_{—}996532, NP_{—}996533, NP_{—}996534, NP_{—}996535, and NP_{—}996537 disclose exemplary human MOG protein sequences, all of which are incorporated by reference as provided by GenBank on Jan. 28, 2011. Similarly, GenBank Accession No. NM_{—}010814 discloses an exemplary mouse MOG nucleic acid sequence, and GenBank Accession No. NP_{—}034944 discloses an exemplary mouse MOG protein sequence, both of which are incorporated by reference as provided by GenBank on Jan. 28, 2011. One of skill in the art can identify additional MOG sequences from human, mouse, or other species.

Pharmaceutical agent or drug: A chemical compound or composition capable of inducing a desired therapeutic or prophylactic effect when properly administered to a subject.

Pharmaceutically acceptable carriers: The pharmaceutically acceptable carriers useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the proteins herein disclosed.

Proteolipid protein (PLP): A myelin protein which is the predominant myelin protein in the central nervous system. PLP is a transmembrane protein. Nucleic acid and protein sequences for PLP are publicly available. For example, GenBank Accession Nos. NM_{—}000533, NM_{—}001128834, and NM_{—}199478 disclose exemplary human PLP nucleic acid sequences, and GenBank Accession Nos. NP_{—}000524, NP_{—}001122306, and NP_{—}955772 disclose exemplary human PLP protein sequences, all of which are incorporated by reference as provided by GenBank on Jan. 28, 2011. Similarly, GenBank Accession No. NM_{—}011123 discloses an exemplary mouse PLP nucleic acid sequence, and GenBank Accession No. NP_{—}035253 discloses an exemplary mouse PLP protein sequence, both of which are incorporated by reference as provided by GenBank on Jan. 28, 2011. One of skill in the art can identify additional PLP sequences from human, mouse, or other species.

Purified: The term purified does not require absolute purity; rather, it is intended as a relative term. Thus, for example, a purified peptide preparation is one in which the peptide or protein is more enriched than the peptide or protein is in its natural environment, for example within a cell. Preferably, a preparation is purified such that the protein or peptide represents at least 50% of the total peptide or protein content of the preparation. In some embodiments, a purified preparation contains at least 60%, at least 70%, at least 80%, at least 85%, at least 90%, at least 95% or more of the protein or peptide.

Recombinant: A recombinant nucleic acid or polypeptide is one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques.

Specific binding agent: An agent that binds substantially only to a defined target. Thus a CD20-specific binding agent binds substantially only the CD20 molecule, or a component thereof. As used herein, the term “CD20-specific binding agent” includes anti-CD20 antibodies and other agents that bind substantially only to CD20 or an epitope thereof.

Anti-CD20 antibodies may be produced using standard procedures described in a number of texts, including Harlow and Lane (Using Antibodies, A Laboratory Manual, CSHL, New York, 1999, ISBN 0-87969-544-7). In addition, certain techniques may enhance the production of neutralizing antibodies (U.S. Pat. No. 5,843,454; U.S. Pat. No. 5,695,927; U.S. Pat. No. 5,643,756; and U.S. Pat. No. 5,013,548). The determination that a particular agent binds substantially only to CD20 may readily be made by using or adapting routine procedures. One suitable in vitro assay makes use of the Western blotting procedure (described in many standard texts, including Harlow and Lane, 1999). Western blotting may be used to determine that a given protein binding agent, such as an anti-CD20 monoclonal antibody, binds substantially only to CD20. Antibodies to CD20 are well known in the art.

Shorter fragments of antibodies can also serve as specific binding agents. For instance, Fabs, Fvs, and single-chain Fvs (scFvs) that bind to CD20 would be CD20-specific binding agents.

Subject: Living multi-cellular vertebrate organisms, a category that includes both human and non-human mammals.

Symptom and sign: A “symptom” is any subjective evidence of disease or of a subject's condition, e.g., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to any measurable parameters such as tests for immunological status or the presence of lesions in a subject with multiple sclerosis, and the presence of joint inflammation and pain in subjects with arthritis.

Systemic Lupus Erythematosus (SLE): An autoimmune disease caused by recurrent injuries to blood vessels in multiple organs, including the kidney, skin, and joints. In patients with SLE, a faulty interaction between T cells and B-cells results in the production of autoantibodies that attack the cell nucleus. There is general agreement that autoantibodies are responsible for at least some aspects of SLE. It is contemplated that new therapies that deplete the B-cell lineage, allowing the immune system to reset as new B-cells are generated from precursors, would offer hope for long lasting benefit in SLE patients.

Therapeutically effective amount: A dose or quantity of a specified compound sufficient to inhibit advancement, or to cause regression of a disease or condition, or which is capable of relieving symptoms caused by the disease or condition. For instance, this can be the amount or dose of a disclosed MHC molecule required to treat or inhibit an autoimmune disorder, such as multiple sclerosis or an inflammatory arthropathy. In one embodiment, a therapeutically effective amount is the amount that, together with an antibody that specifically binds CD20 and optionally one or more additional therapeutic agents (such as additional agents for treating inflammation), induces the desired response in a subject, such as decreasing inflammation. The preparations disclosed herein are administered in therapeutically effective amounts.

Vimentin: A member of the intermediate filament family (e.g., GenBank Gene ID: 7431). Intermediate filaments are part of the cytoskeleton. Vimentin is involved in maintaining cell shape, cytoplasm integrity, and stabilizing cytoskeletal interactions. It is also involved in the immune response and controls transport of low-density lipoprotein-derived cholesterol from a lysosome to the site of esterification.

Nucleic acid and protein sequences for vimentin are publicly available. For example, GenBank Accession No. NM_{—}003380 discloses an exemplary human vimentin nucleic acid sequence, and GenBank Accession No. NP_{—}003371 discloses an exemplary human vimentin protein sequence, both of which are incorporated by reference as provided by GenBank on Jan. 28, 2011. Similarly, GenBank Accession No. NM_{—}011701 discloses an exemplary mouse vimentin nucleic acid sequence, and GenBank Accession No. NP_{—}035831 discloses an exemplary mouse vimentin protein sequence, both of which are incorporated by reference as provided by GenBank on Jan. 28, 2011. One of skill in the art can identify additional vimentin sequences from human, mouse, or other species.

### III. ANTIBODIES THAT BIND TO B CELLS

The methods disclosed herein include administering to a subject one or more antibodies that bind to B cells (for example, in combination with one or more RTLs, such as those disclosed herein). This includes antibodies that specifically bind to a protein expressed by a B cell, including, but not limited to CD20, CD22, CD19, CD40, CD80, and BLyS. In one embodiment, the methods include administering to a subject an antibody that specifically binds CD20 or an antigen binding fragment thereof, such as a monoclonal antibody that specifically binds CD20. Antibodies that specifically bind CD20 are known in the art and are discussed below. In other embodiments, the methods include administering to a subject an antibody (such as a monoclonal antibody) that specifically binds to CD22 (e.g., epratuzumab, bectumomab, inotuzumab, or moxetumomab), CD19 (e.g., blinatumomab or taplitumomab), CD40 (e.g., teneliximab, toralizumab, dacetuzumab, lucatumumab, or ruplizumab), CD80 (e.g., galiximab), BLyS (e.g., belimumab or tabalumab), or an antigen binding fragment thereof. Such antibodies are known to one of skill in the art. See, e.g., Baker et al., Arthritis Rheumatism 48:3253-3265, 2003; Carnahan et al., Cancer Res. 9:3982s-3994s, 2003; U.S. Pat. Nos. 5,874,082; 6,893,638; and 7,462,352; each of which is incorporated by reference herein.

In rheumatoid arthritis, major cell types responsible for chronic inflammation and subsequent cartilage destruction and bone erosion in the joints are macrophages, synovial fibroblasts, neutrophils, and lymphocytes (Marrack et al., Nat. Med. 7:899-905, 2001). It has been demonstrated that T and B lymphocytes that infiltrate inflamed synovial tissues are often organized into structures that resemble lymphoid follicles (Berek & Kim, Semin. Immunol. 9:261-268, 1997; Berek &, Schroder, Ann. NY Acad. Sci. 815:211-217, 1997; Kim & Berek, Arthritis Res. 2:126-131, 2000). Molecular analysis of B cells isolated from synovial follicular structures during rheumatoid arthritis demonstrated the importance of B cells in local antigen-driven specific immune responses and in increased production of rheumatoid factor (RF), the high-affinity antibodies with self-reactivity (Weyand & Goronzy, Ann. NY Acad. Sci. 987:140-149, 2003; Gause et al, BioDrugs 15:73-79, 2001). Positivity for RF is associated with more aggressive articular disease and a higher frequency of extra-articular manifestations (van Zeben et al., Ann. Rheum. Dis. 51:1029-1035, 1992).

In some embodiments, the methods utilize mouse monoclonal, chimeric, humanized, or fully human monoclonal antibodies that bind to B cells. A major limitation in the clinical use of mouse monoclonal antibodies is the development of a human anti-murine antibody (HAMA) response in the patients receiving the treatments. The HAMA response can involve allergic reactions and an increased rate of clearance of the administered antibody from the serum. Various types of modified monoclonal antibodies have been developed to minimize the HAMA response while trying to maintain the antigen binding affinity of the parent monoclonal antibody. One type of modified monoclonal antibody is a human-mouse chimera in which a murine antigen-binding variable region is coupled to a human constant domain (Morrison and Schlom, Important Advances in Oncology, Rosenberg, S. A. (Ed.), 1989). A second type of modified monoclonal antibody is the complementarity determining region (CDR)-grafted, or humanized, monoclonal antibody (Winter and Harris, Immunol. Today 14:243-246, 1993). Fully human antibodies are antibodies wherein the framework region and the CDRs are derived from human sequences. Thus, a HAMA response is not induced when these antibodies are administered to a human subject.

The monoclonal antibody can be of any isotype. The monoclonal antibody can be, for example, an IgM or an IgG antibody, such as IgG₁ or an IgG₂. The class of an antibody that binds to a B cell (such as an antibody that specifically binds CD20, CD22, CD19, CD40, CD80, or BLyS) can be switched with another. In one aspect, a nucleic acid molecule encoding V_{L} or V_{H} is isolated using methods well-known in the art, such that it does not include any nucleic acid sequences encoding the constant region of the light or heavy chain, respectively. The nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that was originally IgM may be class switched to an IgG. Class switching can be used to convert one IgG subclass to another, such as from IgG₁ to IgG₂.

In one embodiment, the antibody that binds to a B cell is fully human. Examples of framework sequences that can be used include the amino acid framework sequences of the heavy and light chains disclosed in PCT Publication No. WO 2006/074071 (see, for example, SEQ ID NOs: 1-16 therein), which is herein incorporated by reference.

Antibody fragments are encompassed by the present disclosure, such as Fab, F(ab′)₂, and Fv which include a heavy chain and light chain variable region and specifically bind a protein expressed by B cells (such as CD20, CD22, CD 19, CD40, CD80, BLyS, or an antigen binding fragment thereof). These antibody fragments retain the ability to specifically bind with the antigen. Fragments of antibodies include scFv, diabodies (scFv dimers), minibodies (scFv-CH₃ dimers), and scFv-Fc (scFv-CH₂—CH₃ dimers). The antibodies can be monovalent or divalent. Methods of making these fragments are known in the art (see for example, Harlow and Lane, Using Antibodies: A Laboratory Manual, Cold Spring Harbor Laboratory Press, 1999).

In a further group of embodiments, the antibodies are Fv antibodies, which are typically about 25 kDa and contain a complete antigen-binding site with three CDRs per each heavy chain and each light chain. To produce these antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; and Pack et al., Bio/Technology 11:1271, 1993). Dimers of a single chain antibody (scFV₂), are also contemplated.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Meth. Enzymol. Vol. 1, page 422, Academic Press, 1967; and Coligan et al. Current Protocols in Immunology, John Wiley & Sons, 2002).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

One of skill will realize that conservative variants of the antibodies can be produced. Such conservative variants employed in antibody fragments, such as dsFv fragments or in scFv fragments, retain critical amino acid residues necessary for correct folding and stabilizing between the V_{H} and the V_{L} regions, and retain the charge characteristics of the residues in order to preserve the low pI and low toxicity of the molecules. Amino acid substitutions (such as at most one, at most two, at most three, at most four, or at most five amino acid substitutions) can be made in the V_{H} and the V_{L} regions to increase yield. Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

A. Antibodies that Specifically Bind CD20

The CD20 molecule (cluster of differentiation 20, also called human B-lymphocyte-restricted differentiation antigen or Bp35) is a hydrophobic transmembrane protein with a molecular weight of approximately 35 kD located on pre-B and mature B lymphocytes (Valentine et al. (1989) J. Biol. Chem. 264(19):11282-11287; and Einfield et al. (1988) EMBO J. 7(3):711-717). CD20 is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin's lymphomas (NHL) (Anderson et al. (1984) Blood 63(6):1424-1433), but is not found on hematopoietic stem cells, pro-B cells, normal plasma cells, or other normal tissues (Tedder et al. (1985) J. Immunol. 135(2):973-979). The 85 amino acid carboxyl-terminal region of the CD20 protein is located within the cytoplasm. An exemplary CD20 sequence is provided as GenBank Accession No. NP_{—}690605 (incorporated herein by reference as present in GenBank on Jan. 28, 2011). It has been proposed that CD20 is involved in regulating early steps in the activation and differentiation process of B cells (Tedder et al. (1986) Eur. J. Immunol. 16:881-887) and could function as a calcium ion channel (Tedder et al. (1990) J. Cell. Biochem. 14D:195).

Certain anti-CD20 monoclonal antibodies can affect the viability and growth of B-cells (Clark et al., Proc. Natl. Acad. Sci. USA 83:4494-98, 1986). Extensive cross-linking of CD20 can induce apoptosis in B lymphoma cell lines (Shan et al., Blood 91:1644-52, 1998), and cross-linking of CD20 on the cell surface has been reported to increase the magnitude and enhance the kinetics of signal transduction, for example, as detected by measuring tyrosine phosphorylation of cellular substrates (Deans et al., J. Immunol. 146:846-53, 1993). Therefore, in addition to cellular depletion by complement and antibody-dependent cell-mediated toxicity (ADCC) mechanisms, Fc-receptor binding by certain CD20 monoclonal antibodies in vivo promotes apoptosis of malignant B-cells by CD20 cross-linking. The presence of multiple membrane spanning domains in the CD20 polypeptide (Einfeld et al., EMBO J. 7:711-17, 1988; Stamenkovic et al., J. Exp. Med. 167:1975-80, 1988; Tedder et al., J. Immunol. 141:4388-4394, 1988), prevent CD20 internalization after antibody binding.

Because normal mature B-cells also express CD20, normal B-cells are depleted by anti-CD20 antibody therapy (Reff et al., Blood 83:435-445, 1994). After treatment is completed, however, normal B-cells can be regenerated from CD20-negative B-cell precursors; therefore, patients treated with anti-CD20 therapy do not experience significant immunosuppression.

In some examples, the presently disclosed methods utilize antibodies or antigen binding fragments thereof that specifically bind CD20, a leukocyte antigen (see, e.g., Stashenko et al, J. Immunol. 125:1678-1685, 1980; Stashenko et al, Proc. Natl. Acad. Sci. USA 78:6 3848, 1981). In some embodiments, the antibody is a monoclonal antibody. The antibody can be a fully human, humanized or a chimeric antibody. However, other antibody forms, such as camelids can be used in the methods disclosed herein. Generally, these antibodies specifically bind CD20. An exemplary amino acid sequence for human CD20 is provided below:

(SEQ ID NO: 13)
MTTPRNSVNG TFPAEPMKGP IAMQSGPKPL FRRMSSLVGP
TQSFFMRESK TLGAVQIMNG LFHIALGGLL MIPAGIYAPI
CVTVWYPLWG GIMYIISGSL LAATEKNSRK CLVKGKMIMN
SLSLFAAISG MILSIMDILN IKISHFLKME SLNFIRAHTP
YINIYNCEPA NPSEKNSPST QYCYSIQSLF LGILSVMLIF
AFFQELVIAG IVENEWKRTC SRPKSNIVLL SAEEKKEQTI
EIKEEVVGLT ETSSQPKNEE DIEIIPIQEE EEEETETNFP
EPPQDQESSP IENDSSP

See also GenBank Accession No. NP_{—}068769, as of Jan. 28, 2011, incorporated by reference herein.

Antibodies that specifically bind CD20 are commercially available and are known in the art. For example, antibodies that bind CD20 are disclosed in U.S. Pat. No. 7,850,962, which is incorporated herein by reference. Such antibodies include antibodies (and antigen binding fragments derived from the antibodies) known as RITUXAN® (rituximab), ZEVALIN® (ibritumomab tiuxetan), and BEXXAR® (tositumomab) (see, e.g., U.S. Pat. Nos. 5,595,721, 5,843,398, 6,015,542, 6,090,365, 6,565,827, 6,287,537; 6,399,061; 6,455,043; 6,682,734; and U.S. Pat. No. 5,736,137). Ofatumumab (ARZERRA®), ocrelizumab, and afutuzumab also specifically bind CD20. Additional antibodies that bind CD20 are disclosed, for example, in U.S. Patent Publication Nos. 2009/0203886 and 2010/0330089. Further antibodies that bind CD20 include veltuzumab (U.S. Pat. No. 7,919,273) and FBTA05 (Stanglmaier et al., Int. J. Cancer 123:1181-1189, 2008).

In one non-limiting example, the antibody that specifically binds CD20 is rituximab. Rituximab is a recombinant mouse human IgG₁ chimeric mAb in which variable domains of the heavy and light chains of a murine anti-CD20 mAb were fused to the human constant regions of IgG1. Rituximab was tested for safety, tolerability and preliminary clinical efficacy for the treatment of 18 patients with Systemic Lupus Erythematosus (SLE) (who were non-immunosuppressed patients). The antibodies are preferably humanized or human.

U.S. Pat. No. 5,736,137 (incorporated by reference herein) discloses that effective dosages (e.g., therapeutically effective amounts) of the immunologically active chimeric anti-CD20 antibodies range from about 0.001 to about 30 mg/kg body weight, such as from about 0.01 to about 25 mg/kg body weight, for example from about 0.4 to about 20.0 mg/kg body weight. Exemplary doses of rituximab are 100 mg/m² (low dose) and 375 mg/m² (medium dose), administered either once or several times at weekly intervals. The amount of the antibody that specifically binds CD20 can vary according to the size of the individual to whom the therapy is being administered, as well as the characteristics of the disorder being treated. In exemplary treatments, about 1 mg/day, about 5 mg/day, about 10 mg/day, about 20 mg/day, about 50 mg/day, about 75 mg/day, about 100 mg/day, about 150 mg/day, about 200 mg/day, about 250 mg/day, about 400 mg/day, about 500 mg/day, about 800 mg/day, about 1000 mg/day, about 1600 mg/day or about 2000 mg/day is administered. The doses may also be administered based on weight of the patient, e.g., at a dose of 0.01 to 50 mg/kg. In a related embodiment, the antibody that specifically binds CD20 can be administered in a dose range of 0.015 to 30 mg/kg. In an additional embodiment, the antibody that specifically binds CD20 is administered in a dose of about 0.015, about 0.05, about 0.15, about 0.5, about 1.5, about 5, about 15 or about 30 mg/kg. Other dosages can be used; factors influencing dosage include, but are not limited to, the severity of the disease, previous treatment approaches, overall health of the patient, other diseases present, etc. One of skill in the art can readily determine a suitable dosage that falls within the ranges, or if necessary, outside of the ranges.

Introduction of the immunologically active anti-CD20 antibodies in these dose ranges can be carried out as a single treatment or over a series of treatments. With respect to these antibodies, in some embodiments they are administered to a subject over a series of treatments. U.S. Pat. No. 5,736,137 discloses that, while without being bound by any particular theory, because the immunologically active chimeric anti-CD20 antibodies are both immunologically active and bind to CD20, upon initial introduction of the immunologically active chimeric anti-CD20 antibodies to the individual, peripheral blood B cell depletion will begin. A nearly complete depletion was observed within about 24 hours post treatment infusion. Subsequent introduction(s) of the immunologically active chimeric anti-CD20 antibodies (or radiolabeled anti-CD20 antibodies) to the patient is presumed to: a) clear remaining peripheral blood B cells; b) begin B cell depletion from lymph nodes; c) begin B cell depletion from other tissue sources. Thus, repeated introduction of the anti-CD20 antibodies results in substantially depleting the patient's peripheral blood B cells and clearing remaining B cells from the system clearing lymph node B cells, or clearing other tissue B cells. One treatment course can occur over several stages; most preferably, between about 0.4 and about 20 mg/kg body weight of the immunologically active chimeric anti-CD20 antibodies is introduced to the patient once a week for between about 2 to 10 weeks, most preferably for about 4 weeks. However, a single dosage provides benefits and can be effectively utilized for disease treatment/management.

Evidence regarding the pathogenicity of B cells in RA has been recently obtained from clinical trials in patients with refractory disease by using B cell ablation with rituximab (Leandro et al, Ann. Rheum. Dis. 61:883-888, 2002; Edwards et al., N. Engl. J. Med. 350:2572-2581, 2004, incorporated herein by reference). In all groups treated with rituximab, a significantly higher proportion of patients had a 20 percent improvement in disease symptoms according to the ACR criteria. All ACR responses were maintained at week 48 in the rituximab-methotrexate group. In this study involving 161 patients with active RA, serious infections occurred in one patient (2.5 percent) in the control group and in four patients (3.3 percent) in the rituximab groups, indicating that B cell depletion is a relatively safe therapy in RA.

### IV. RTLS

The disclosed methods utilize RTLs in methods of treatment of an autoimmune disorder, such as, but not limited to, rheumatoid arthritis or multiple sclerosis. RTLs are monomeric recombinant polypeptides that can mimic MHC function and include only those MHC domains that define an antigen binding cleft. The RTLs are capable of antigen-specific T-cell binding and include, in the case of human class II MHC molecules, only the α1 and β1 domains in covalent linkage (and in some examples in association with an antigenic determinant). For convenience, such MHC class II polypeptides are referred to herein as “β1α1.” Equivalent molecules derived from human MHC class I molecules are also provided herein. Such molecules comprise the α1 and α2 domains of class I molecules in covalent linkage (and in some examples in association with an antigenic determinant). Such MHC class I polypeptides are referred to herein as “α1α2.” These two domain molecules may be readily produced by recombinant expression in prokaryotic or eukaryotic cells, and readily purified in large quantities. Moreover, these molecules may easily be loaded with any desired peptide antigen, making production of a repertoire of MHC molecules with different T-cell specificities a simple task.

A. Recombinant MHC Class II Pα1 Molecules

The amino acid sequences of mammalian MHC class II α and β chain proteins, as well as nucleic acids encoding these proteins, are well known in the art and available from numerous sources including GenBank. Exemplary sequences are provided in Auffray et al. (Nature 308:327-333, 1984) (human HLA DQ α); Larhammar et al. (Proc. Natl. Acad. Sci. USA 80:7313-7317, 1983) (human HLA DQ β); Das et al. (Proc. Natl. Acad. Sci. USA 80:3543-3547, 1983) (human HLA DR α); Tonnelle et al. (EMBO J. 4:2839-2847, 1985) (human HLA DR β); Lawrance et al. (Nucl. Acids Res. 13:7515-7528, 1985) (human HLA DP α); Kelly and Trowsdale (Nucl. Acids Res. 13:1607-1621, 1985) (human HLA DP β); Syha et al. (Nucl. Acids Res. 17:3985, 1989) (rat RT1.B α); Syha-Jedelhauser et al. (Biochim. Biophys. Acta 1089:414-416, 1991) (rat RT1.B β); Benoist et al. (Proc. Natl. Acad. Sci. USA 80:534-538, 1983) (mouse I-A α); Estess et al. (Proc. Natl. Acad. Sci. USA 83:3594-3598, 1986) (mouse I-A β), all of which are incorporated by reference herein. In one embodiment, the MHC class II protein is a human MHC class II protein (such as HLA-DR, HLA-DQ, or HLA-DP). In a particular embodiment, the MHC class II protein is a human HLA-DR, such as HLA-DR4.

The recombinant MHC class II molecules (e.g., RTLs) of the present disclosure include the β1 domain of the MHC class II β chain covalently linked to the α1 domain of the MHC class II α chain. The α1 and β1 domains are well defined in mammalian MHC class II proteins. In some examples, MHC class II α chains include a leader sequence that is involved in trafficking the polypeptide and is proteolytically removed to produce the mature a polypeptide. Typically, the α1 domain is regarded as comprising about residues 1-90 of the mature chain. The native peptide linker region between the α1 and α2 domains of the MHC class II protein spans from about amino acid 76 to about amino acid 93 of the mature a chain, depending on the particular α chain under consideration. Thus, an α1 domain may include about amino acid residues 1-90 of the mature α chain, but one of skill in the art will recognize that the C-terminal cut-off of this domain is not necessarily precisely defined, and, for example, might occur at any point between amino acid residues 70-100 of the α chain. In some examples, the α1 domain includes amino acids 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of a mature MHC class II α domain. In other examples, an α1 domain includes about residues 20-120 (such as about residues 20-110, 24-110, 24-109, 25-100, 25-109, 26-110, 26-109, 30-120, 32-120, 32-115, 26-90, 26-85, 26-84, or other overlapping regions) of the full length MHC class II α polypeptide. In some examples, the MHC class II α1 domain does not include an N-terminal methionine; however, an N-terminal methionine can be present, for example as a result of expression in a bacterial, yeast, or mammalian system. The composition of the α1 domain may also vary outside of these parameters depending on the mammalian species and the particular α chain in question. One of skill in the art will appreciate that the precise numerical parameters of the amino acid sequence are less important than the maintenance of domain function.

Similarly, the β1 domain is typically regarded as comprising about residues 1-90 of the mature β chain. The linker region between the β1 and β2 domains of the MHC class II protein spans from about amino acid 85 to about amino acid 100 of the β chain, depending on the particular β chain under consideration. Thus, the β1 protein may include about amino acid residues 1-100, but one of skill in the art will again recognize that the C-terminal cut-off of this domain is not necessarily precisely defined, and, for example, might occur at any point between amino acid residues 75-105 of the β chain. In some examples, the β1 domain includes amino acids 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of a mature MHC class II β chain. In some examples, the MHC class II β1 domain does not include an N-terminal methionine; however, an N-terminal methionine can be present, for example as a result of expression in a bacterial, yeast, or mammalian system. The composition of the β1 domain may also vary outside of these parameters depending on the mammalian species and the particular β chain in question. Again, one of skill in the art will appreciate that the precise numerical parameters of the amino acid sequence are less important than the maintenance of domain function. In one embodiment, the β1α1 molecules do not include a β2 domain. In another embodiment, the β1α1 molecules do not include an α2 domain. In yet a further embodiment, the β1α1 molecules do not include either an α2 or a β2 domain.

Nucleic acid molecules encoding these domains may be produced by standard means, such as amplification by the polymerase chain reaction (PCR). Standard approaches for designing primers for amplifying open reading frames encoding these, domains may be employed. Libraries suitable for the amplification of these domains include, for example, cDNA libraries prepared from the mammalian species in question; such libraries are available commercially, or may be prepared by standard methods. Thus, for example, constructs encoding the β1 and α1 polypeptides may be produced by PCR using four primers: primers B1 and B2 corresponding to the 5′ and 3′ ends of the β1 coding region, and primers A1 and A2 corresponding to the 5′ and 3′ ends of the α1 coding region. Following PCR amplification of the β1 and α1 domain coding regions, these amplified nucleic acid molecules may each be cloned into standard cloning vectors, or the molecules may be ligated together and then cloned into a suitable vector. To facilitate convenient cloning of the two coding regions, restriction endonuclease recognition sites may be designed into the PCR primers. For example, primers B2 and A1 may each include a suitable site such that the amplified fragments may be readily ligated together following amplification and digestion with the selected restriction enzyme. In addition, primers B1 and A2 may each include restriction sites to facilitate cloning into the polylinker site of the selected vector. Ligation of the two domain coding regions is performed such that the coding regions are operably linked, e.g., to maintain the open reading frame. Where the amplified coding regions are separately cloned, the fragments may be subsequently released from the cloning vector and gel purified, preparatory to ligation.

In certain embodiments, a peptide linker is provided between the β1 and a 1 domains. Typically, this linker is between 2 and 25 amino acids in length, and serves to provide flexibility between the domains such that each domain is free to fold into its native conformation. The linker sequence may conveniently be provided by designing the PCR primers to encode the linker sequence. Thus, in the example described above, the linker sequence may be encoded by one of the B2 or A1 primers, or a combination of each of these primers.

Exemplary MHC class I polypeptides are disclosed in U.S. Pat. No. 6,270,772 and U.S. Pat. Application Publication Nos. 2005/0142142, 2008/0267987, and 2009/0280135; each of which is incorporated by reference in their entirety. In a particular example, an MHC class II β1α1 molecule suitable for use in the disclosed methods is RTL362 (e.g., SEQ ID NO: 2). This RTL includes covalently linked β1 and α1 domains of human HLA-DR4 covalently linked via a peptide linker to human collagen II 261-273 peptide (e.g., SEQ ID NO: 4) or human collagen II 259-273 peptide (e.g., SEQ ID NO: 6). These collagen II peptides can be replaced with one or more different antigens, such as those disclosed below. The disclosed β1α1 polypeptides may include an N-terminal methionine (for example, as in SEQ ID NOs: 2, 8, 14, and 42); however, the N-terminal methionine is not required and the β1α1 polypeptide may be synthesized without this residue, or it may be subsequently removed prior to use.

B. Recombinant MHC Class I α1α2 Molecules

The amino acid sequences of mammalian MHC class I α chain proteins, as well as nucleic acids encoding these proteins, are well known in the art and available from numerous sources including GenBank. Exemplary sequences are provided in Browning et al. (Tissue Antigens 45:177-187, 1995) (human HLA-A); Kato et al. (Immunogenetics 37:212-216, 1993) (human HLA-B); Steinle et al. (Tissue Antigens 39:134-137, 1992) (human HLA-C); Walter et al. (Immunogenetics 41:332, 1995) (rat Ia); Walter et al. (Immunogenetics 39:351-354, 1994) (rat Ib); Kress et al. (Nature 306:602-604, 1983) (mouse H-2-K); Schepart et al. (J. Immunol. 136:3489-3495, 1986) (mouse H-2-D); and Moore et al. (Science 215:679-682, 1982) (mouse H-2-1), which are incorporated by reference herein. In one embodiment, the MHC class I protein is a human MHC class I protein.

The recombinant MHC class I molecules of the present disclosure comprise the α1 domain of the MHC class I α chain covalently linked to the α2 domain of the MHC class I chain. These two domains are well defined in mammalian MHC class I proteins. Typically, the α1 domain is regarded as comprising about residues 1-90 of the mature chain and the α2 chain as comprising about amino acid residues 90-180, although again, the cut-off points are not precisely defined and will vary between different MHC class I molecules. The boundary between the α2 and α3 domains of the MHC class I α protein typically occurs in the region of amino acids 179-183 of the mature chain. The composition of the α1 and α2 domains may also vary outside of these parameters depending on the mammalian species and the particular α chain in question. One of skill in the art will appreciate that the precise numerical parameters of the amino acid sequence are less important than the maintenance of domain function. In one embodiment, the α1α2 molecule does not include an α3 domain.

The α1α2 construct may be most conveniently constructed by amplifying the reading frame encoding the dual-domain (α1 and α2) region between amino acid number 1 and amino acids 179-183, although one of skill in the art will appreciate that some variation in these end-points is possible. Such a molecule includes the native linker region between the α1 and α2 domains, but if desired that linker region may be removed and replaced with a synthetic linker peptide. The general considerations for amplifying and cloning the MHC class I α1 and α2 domains apply as discussed above in the context of the MHC class II β1 and α1 domains.

Exemplary MHC class I α1α2 polypeptides are disclosed in U.S. Pat. No. 7,265,218 and U.S. Pat. Application Publication Nos. 2005/0142142, 2008/0267987, and 2009/0280135; each of which is incorporated by reference in their entirety.

C. Modified MHC Molecules

While the foregoing discussion uses as examples naturally occurring MHC class I and class II molecules and the various domains of these molecules, one of skill in the art will appreciate that variants of these molecules and domains may be made and utilized in the same manner as described. Thus, reference herein to a domain of an MHC polypeptide or molecule (e.g., an MHC class II β1 domain) includes both naturally occurring forms of the referenced molecule, as well as molecules that are based on the amino acid sequence of the naturally occurring form, but which include one or more amino acid sequence variations. Such variant polypeptides may also be defined in the degree of amino acid sequence identity that they share with the naturally occurring molecule. Typically, MHC domain variants will share at least 80% sequence identity with the sequence of the naturally occurring MHC domain. More highly conserved variants will share at least 90% or at least 95% sequence identity with the naturally occurring sequence. Variants of MHC domain polypeptides also retain the biological activity of the naturally occurring polypeptide. For the purposes of this disclosure, that activity is conveniently assessed by incorporating the variant domain in the appropriate β1α1 or α1α2 polypeptide and determining the ability of the resulting polypeptide to inhibit antigen specific T-cell proliferation in vitro.

Methods of determining antigen-specific T-cell proliferation are well known to one of skill in the art (see, e.g., Huan et al., J. Chem. Technol. Biotechnol. 80:2-12, 2005). In one example, T cells and APCs are incubated with stimulation medium only, Con A, or antigen with or without supplemental IL-2 (20 Units/ml) at 37° C. in 7% CO₂. The cultures are incubated for three days, the last 18 hours in the presence of [³H]thymidine. The cells are harvested and [³H]thymidine uptake assessed (for example by liquid scintillation counting).

Variant MHC domain polypeptides include proteins that differ in amino acid sequence from the naturally occurring MHC polypeptide sequence but which retain the specified biological activity. Such proteins may be produced by manipulating the nucleotide sequence of the molecule encoding the domain, for example by site-directed mutagenesis or the polymerase chain reaction. The simplest modifications involve the substitution of one or more amino acids for amino acids having similar biochemical properties. These so-called conservative substitutions are likely to have minimal impact on the activity of the resultant protein. Table 1 shows examples of amino acids which may be substituted for an original amino acid in a protein and which are regarded as conservative substitutions.

TABLE 1
Exemplary conservative amino acid substitutions

| Original Amino Acid | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

More substantial changes in biological function or other features may be obtained by selecting substitutions that are less conservative than those shown above, e.g., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine. The effects of these amino acid substitutions or deletions or additions may be assessed through the use of the described T-cell proliferation assay.

At the nucleic acid level, one of skill in the art will appreciate that the naturally occurring nucleic acid sequences that encode class I and II MHC domains may be employed in the expression vectors, but that the disclosure is not limited to such sequences. Any sequence that encodes a functional MHC domain may be employed, and the nucleic acid sequence may be adapted to conform with the codon usage bias of the organism in which the sequence is to be expressed.

In some embodiments, the disclosed MHC molecules include modified MHC molecules that include one or more amino acid changes that decrease self-aggregation of native MHC polypeptides or β1α1 or α1α2 MHC molecules.

Typically, modified MHC molecules of the disclosure are rationally designed and constructed to introduce one or more amino acid changes at a solvent-exposed target site located within, or defining, a self-binding interface found in the native MHC polypeptide. The self-binding interface that is altered in the modified MHC molecule typically includes one or more amino acid residues that mediate self-aggregation of a native MHC polypeptide, or of an “unmodified” β1α1 or α1α2 MHC molecule incorporating the native MHC polypeptide. Although the self-binding interface is correlated with the primary structure of the native MHC polypeptide, this interface may only appear as an aggregation-promoting surface feature when the native polypeptide is isolated from the intact MHC complex and incorporated in the context of an “unmodified” β1α1 or α1α2 MHC molecule. In the case of exemplary MHC class II molecules described herein (e.g., comprising linked β1 and α1 domains), the native β1α1 structure only exhibits certain solvent-exposed, self-binding residues or motifs after removal of Ig-fold like β2 and α2 domains found in the intact MHC II complex. These same residues or motifs that mediate aggregation of unmodified β1α1 MHC molecules, are presumptively “buried” in a solvent-inaccessible conformation or otherwise “masked” (e.g., prevented from mediating self-association) in the native or progenitor MHC II complex (likely through association with the Ig-fold like β2 and α2 domains).

In some examples, an MHC molecule which has a reduced potential for aggregation in solution includes an “MHC component” in the form of a single chain polypeptide that includes multiple, covalently-linked MHC domain elements. These domain elements are typically selected from a) α1 and β1 domains of an MHC class II polypeptide, or portions thereof comprising an Ag-binding groove/T-cell receptor (TCR) interface; or b) α1 and α2 domains of an MHC class I polypeptide, or portions thereof comprising an Ag-binding groove/TCR interface. The MHC component of the molecule is modified by one or more amino acid substitutions, additions, deletions, or rearrangements at a target site corresponding to a “self-binding interface” identified in a native MHC polypeptide component of an unmodified β1α1 or α1α2 MHC molecule. The modified β1α1 or α1α2 MHC molecule exhibits a markedly reduced propensity for aggregation in solution compared to aggregation exhibited by an unmodified, control β1α1 or α1α2 MHC molecule having the same fundamental MHC component structure, but incorporating the native MHC polypeptide defining the self-binding interface. Modified β1α1 or α1α2 MHC molecules with reduced potential for aggregation are described in detail in U.S. Patent Publication No. 2005/0142142, incorporated by reference herein in its entirety.

The modified MHC molecules disclosed herein yield an increased percentage of monodisperse (monomeric) molecules in solution compared to a corresponding, unmodified MHC molecule (e.g., comprising the native MHC polypeptide and bearing the unmodified, self-binding interface). In certain embodiments, the percentage of unmodified MHC molecule present as a monodisperse species in aqueous solution may be as low as 1%, more typically 5-10% or less of total MHC protein, with the balance of the unmodified MHC molecule being found in the form of higher-order aggregates. In contrast, modified MHC molecules disclosed herein yield at least 10%-20% monodisperse species in solution. In other embodiments, the percentage of monomeric species in solution will range from 25%-40%, often 50%-75%, up to 85%, 90%, 95%, or greater of the total MHC protein present, with a commensurate reduction in the percentage of aggregate MHC species compared to quantities observed for the corresponding, unmodified MHC molecules under comparable conditions.

MHC modification typically involves amino acid substitution or deletion at target sites for mutagenesis comprising a self-binding interface (including one or more amino acid residues, or a self-binding motif formed of several target residues). Within exemplary embodiments directed toward production of modified MHC molecule that include MHC class II β1α1 components, targeted residues for modification typically include hydrophobic residues or motifs, for example valine, leucine, isoleucine, alanine, phenylalanine, tyrosine, and tryptophan. These and other target residues may be substituted for any non-hydrophobic amino acid. Suitable amino acids for generating desired MHC molecule modifications can include amino acids having aliphatic-hydroxyl side chains, such as serine and threonine; amino acids having amide-containing side chains, such as asparagine and glutamine; amino acids having aromatic side chains, such as phenylalanine, tyrosine, and tryptophan; and amino acids having basic side chains, such as lysine, arginine, and histidine.

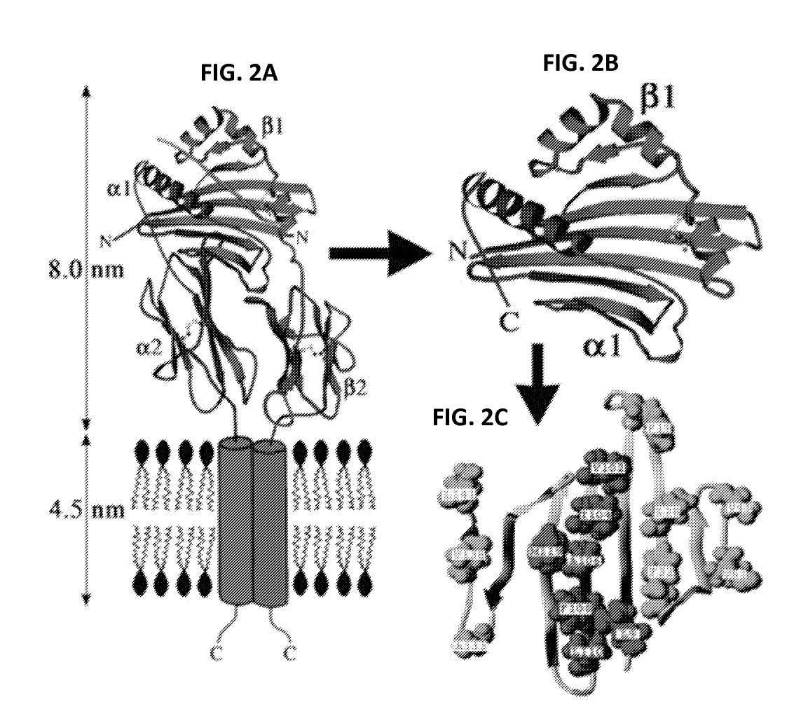

In some examples, surface modification of an MHC molecule comprising an MHC class II component to yield much less aggregation prone form can be achieved, for example, by replacement of one or more hydrophobic residues identified in the β-sheet platform of the MHC component with non-hydrophobic residues, for example polar or charged residues. FIGS. 2A-C depict an exemplary HLA-DR2 polypeptide, an exemplary β1α1 molecule, and hydrophobic β-sheet platform residues that may be targeted for modification. In some examples, one or more hydrophobic amino acids of a central core portion of the β-sheet platform are modified, such as one or more of V102, I104, A106, F108, and L110 of a human MHC class II β1α1 RTL (for example, SEQ ID NO: 14). In some examples, hydrophobic amino acids of a central core portion of the β-sheet platform include one or more amino acids at positions 6, 8, 10, 12, and 14 of an MHC class II α chain polypeptide or α1 domain (such as a mature human MHC class II α polypeptide). In one example the amino acids include one or more of V6, I8, A10, F12, and L14 of a mature human MHC class II α chain, such as a human DR2 polypeptide. One of skill in the art can identify corresponding amino acids in other MHC class II molecules or β1α1 molecules.

In particular examples, one or more of the identified hydrophobic β-sheet platform amino acids is changed to either to a polar (for example, serine) or charged (for example, aspartic acid) residue. In some examples all five of V102, I104, A106, F108, and L110 (or corresponding amino acids in another MHC molecule) are changed to a polar or charged residue. In one example, each of V 102, I104, A106, F108, and L110 are changed to an aspartic acid residue.

In other examples, additional hydrophobic target residues are available for modification to alter self-binding characteristics of the β-sheet platform portion of class II MHC molecules incorporated in MHC molecules. In reference to FIG. 2C, the left arm of the diagrammed β-sheet platform includes a separate “motif” of three noted hydrophobic residues (top to bottom), L141, V138, and A133 of a human MHC class II β1α1RTL (for example SEQ ID NO: 14) that can be modified to a non-hydrophobic (e.g., polar, or charged) residue. Also in reference to FIG. 2C, several target hydrophobic residues are marked to the right of the core β-sheet motif, including L9, F19, L28, F32, V45, and V51 of a human MHC class II β1α1 RTL (for example SEQ ID NO: 14), which may be regarded as one or more additional, self-binding or self-associating target “motifs” for MHC molecule modification. Any one or a combination of these residues may be targeted for modification to a non-hydrophobic residue, increasing monomeric MHC molecules.

D. Expression and Purification of Recombinant MHC Molecules

In some embodiments, the MHC molecules disclosed herein (such as MHC class II β1α1 molecules or MHC class I α1α2 molecules) are expressed in prokaryotic or eukaryotic cells from a nucleic acid construct. In their most basic form, nucleic acids encoding the MHC polypeptides of the disclosure comprise first and second regions, having a structure A-B (or B-A) wherein, for class I molecules, region A encodes the class I α1 domain and region B encodes the class I α2 domain. For class II molecules, A encodes the class II α1 domain and B encodes the class II β1 domain. Where a linker sequence is included, the nucleic acid may be represented as B-L2-A, wherein L2 is a nucleic acid sequence encoding the linker peptide. Where an antigenic peptide is covalently linked to the MHC polypeptide, the nucleic acid molecule encoding this complex may be represented as P-B-A. A second linker sequence may be provided between the antigenic protein and the region B polypeptide, such that the coding sequence is represented as P-L2-B-L1-A. In all instances, the various nucleic acid sequences that comprise the MHC polypeptide (e.g., L1, L2, B, A and P) are operably linked such that the elements are situated in a single reading frame.

Nucleic acid constructs expressing these MHC polypeptides may also include regulatory elements such as promoters (Pr), enhancers, and 3′ regulatory regions, the selection of which will be determined based upon the type of cell in which the protein is to be expressed. When a promoter sequence is operably linked to the open reading frame, the sequence may be represented as Pr-B-A, or (if an antigen-coding region is included) Pr-P-B-A, wherein Pr represents the promoter sequence. The promoter sequence is operably linked to the P or B components of these sequences, and the B-A or P-B-A sequences comprise a single open reading frame. The constructs are introduced into a vector suitable for expressing the MHC polypeptide in the selected cell type.

Numerous prokaryotic and eukaryotic systems are known for the expression and purification of polypeptides. For example, heterologous polypeptides can be produced in prokaryotic cells by placing a strong, regulated promoter and an efficient ribosome binding site upstream of the polypeptide-encoding construct. Suitable promoter sequences include the beta-lactamase, tryptophan (trp), phage T7 and lambda P_{L} promoters. Methods and plasmid vectors for producing heterologous proteins in bacteria or mammalian cells are described in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); and Ausubel et al., Short Protocols in Molecular Biology: A Compendium of Methods from Current Protocols in Molecular Biology, 4^{th} ed., Wiley & Sons, 1999.

Suitable prokaryotic cells for expression of large amounts of fusion proteins include Escherichia coli and Bacillus subtilis. Often, proteins expressed at high levels are found in insoluble inclusion bodies; methods for extracting proteins from these aggregates are described for example, by Sambrook et al. (2001, see chapter 15). Recombinant expression of MHC polypeptides in prokaryotic cells may alternatively be conveniently obtained using commercial systems designed for optimal expression and purification of fusion proteins. Such fusion proteins typically include a protein tag that facilitates purification. Examples of such systems include: the pMAL protein fusion and purification system (New England Biolabs, Inc., Beverly, Mass.); the GST gene fusion system (Amersham Pharmacia Biotech, Inc., Piscataway, N.J.); and the pTrcHis expression vector system (Invitrogen, Carlsbad, Calif.). For example, the pMAL expression system utilizes a vector that adds a maltose binding protein to the expressed protein. The fusion protein is expressed in E. coli. and the fusion protein is purified from a crude cell extract using an amylose column. If necessary, the maltose binding protein domain can be cleaved from the fusion protein by treatment with a suitable protease, such as Factor Xa. The maltose binding fragment can then be removed from the preparation by passage over a second amylose column.

The MHC polypeptides can also be expressed in eukaryotic expression systems, including Pichia pastoris, Drosophila, Baculovirus and Sindbis expression systems produced by Invitrogen (Carlsbad, Calif.). Eukaryotic cells such as Chinese Hamster ovary (CHO), monkey kidney (COS), HeLa, Spodoptera frugiperda, and Saccharomyces cerevisiae may also be used to express the MHC polypeptides. Regulatory regions suitable for use in these cells include, for mammalian cells, viral promoters such as those from CMV, adenovirus or SV40, and for yeast cells, the promoter for 3-phosphoglycerate kinase or alcohol dehydrogenase.

The transfer of DNA into eukaryotic, in particular human or other mammalian cells, is now a conventional technique. The vectors are introduced into the recipient cells as pure DNA (transfection) by, for example, precipitation with calcium phosphate or strontium phosphate, electroporation, lipofection, DEAE dextran, microinjection, protoplast fusion, or microprojectile guns. Alternatively, the nucleic acid molecules can be introduced by infection with virus vectors. Systems are developed that use, for example, retroviruses, adenoviruses, or Herpes virus.

An MHC polypeptide produced in mammalian cells may be extracted following release of the protein into the supernatant and may be purified using an immunoaffinity column prepared using anti-MHC antibodies. Alternatively, the MHC polypeptide may be expressed as a chimeric protein with, for example, β-globin. Antibody to β-globin is thereafter used to purify the chimeric protein. Corresponding protease cleavage sites engineered between the β-globin gene and the nucleic acid sequence encoding the MHC polypeptide are then used to separate the two polypeptide fragments from one another after translation. One useful expression vector for generating β-globin chimeric proteins is pSG5 (Stratagene, La Jolla, Calif.).

Expression of the MHC polypeptides in prokaryotic cells will result in polypeptides that are not glycosylated. Glycosylation of the polypeptides at naturally occurring glycosylation target sites may be achieved by expression of the polypeptides in suitable eukaryotic expression systems, such as mammalian cells.

Purification of the expressed protein is generally performed in a basic solution (typically around pH 10) containing 6M urea. Folding of the purified protein is then achieved by dialysis against a buffered solution at neutral pH (typically phosphate buffered saline at around pH 7.4).

E. Antigens

In some embodiments, the disclosed methods include MHC molecules including a covalently linked antigen. As is well known in the art (see for example U.S. Pat. No. 5,468,481) the presentation of antigen in MHC complexes on the surface of APCs generally does not involve a whole antigenic peptide. Rather, a peptide located in the groove between the β1 and α1 domains (in the case of MHC II) or the α1 and α2 domains (in the case of MHC I) is typically a small fragment of the whole antigenic peptide. As discussed in Janeway & Travers (Immunobiology: The Immune System in Health and Disease, 1997), peptides located in the peptide groove of MHC class I molecules are constrained by the size of the binding pocket and are typically 8-15 amino acids long (such as 8, 9, 10, 11, 12, 13, 14, or 15 amino acids), more typically 8-10 amino acids in length (but see Collins et al., Nature 371:626-629, 1994 for possible exceptions). In contrast, peptides located in the peptide groove of MHC class II molecules are not constrained in this way and are often larger, typically at least 8-50 amino acids in length (such as 8-30, 10-25, or 15-23 amino acids in length). In some examples, the peptide located in the peptide groove of an MHC class II molecule is about 13-23 amino acids in length. In other examples, the peptide is at least about 8, 10, 15, 20, 25, 30, 35, 40, 45, 50, 55, 60, 65, 70, 75, or more amino acids in length. Peptide fragments for loading into MHC molecules can be prepared by standard means, such as use of synthetic peptide synthesis machines.

In some examples, the antigen is glycosylated and/or citrullinated. For example, the antigen may include one or more N-linked glycosylation and/or O-linked glycosylation. In one example, the antigen is collagen II or a portion thereof, and the glycosylation is O-linked glycosylation of a hydroxylysine residue. In other examples, the antigen may include one or more citrulline residue produced by deamination of an arginine residue in the antigen. In some examples, the citrullinated antigen is fibrinogen-α, vimentin, α-enolase, cartilage glycoprotein-39, or a portion thereof.

The antigen is selected based on the condition to be treated. For example, for the treatment of multiple sclerosis, antigens of use include a myelin protein (for example, myelin oligodendrocyte glycoprotein (MOG), myelin basic protein (MBP), or proteolipid protein (PLP)), or an antigenic determinant thereof, wherein the antigenic determinant is 8 to 30 amino acids in length and binds the MHC molecule. Particular antigens include MOG 35-55 (MEVGWYRPPFSRVVHLYRNGK; SEQ ID NO: 15), MOG 1-25 (GQFRVIGPRHPIRALVGDEVELPCR; SEQ ID NO: 16), MOG 94-116 (GGFTCFFRDHSYQEEAAMELKVE; SEQ ID NO: 17), MOG 145-160 (VFLCLQYRLRGKLRAE; SEQ ID NO: 18), MOG 194-208 (LVALIICYNWLHRRL; SEQ ID NO: 19), MBP 10-30 (RHGSKYLATASTMDHARHGFL; SEQ ID NO:20), MBP 35-45 (DTGILDSIGRF; SEQ ID NO: 21), MBP 77-91 (SHGRTQDENPVVHF; SEQ ID NO: 22), MBP 85-99 (ENPVVHFFKNIVTPR; SEQ ID NO: 23), MBP 95-112 (IVTPRTPPPSQGKGRGLS; SEQ ID NO: 24), MBP 145-164 (VDAQGTLSKIFKLGGRDSRS; SEQ ID NO: 25), PLP 139-151 (CHCLGKWLGHPDKFVG; SEQ ID NO: 26), and PLP 95-116 (GAVRQIFGDYKTTICGKGLSAT; SEQ ID NO: 27). One of skill in the art can identify additional myelin protein antigens.

For the treatment of rheumatoid arthritis, the antigen can be collagen, vimentin, fibrinogen-α, α-enolase, human cartilage glycoprotein-39, or an antigenic determinant thereof, wherein the antigenic determinant is 8 to 30 amino acids in length and binds the MHC molecule. In some embodiments, the antigen is a collagen, such as human type II collagen (collagen II). Exemplary antigens of use include, but are not limited to, collagen II 261-274 (AGFKGEQGPKGEPG; SEQ ID NO: 28), collagen II 259-273 (GIAGFKGEQGPKGEP; SEQ ID NO: 29), collagen II 257-270 (EPGIAGFKGEQGPK; SEQ ID NO: 30), or modified collagen II 257-270 (APGIAGFKAEQAAK; SEQ ID NO: 31). In some examples, the collagen II antigen is glycosylated.

In other embodiments, the antigen is fibrinogen-α, such as human fibrinogen-α or an antigenic determinant thereof. Specific examples of antigens of use include, but are not limited to, fibrinogen-α 40-59 (VERHQSACKDSDWPFCSDED; SEQ ID NO: 32), fibrinogen-α 616-625 (THSTKRGHAKSRPVRGIHTS; SEQ ID NO: 33), fibrinogen-α 79-91 (QDFTNRINKLKNS; SEQ ID NO: 34), or fibrinogen-α 121-140 (NNRDNTYNRVSEDLRSRIEV; SEQ ID NO: 35). In some examples, the fibrinogen-α antigen is citrullinated.

In additional embodiments, the antigen is vimentin, such as human vimentin or an antigenic determinant thereof. Specific examples of antigens of use include, but are not limited to, vimentin 59-79 (GVYATRSSAVRLRSSVPGVRL; SEQ ID NO: 36), vimentin 26-44 (SSRSYVTTSTRTYSLGSAL; SEQ ID NO: 37), vimentin 256-275 (IDVDVSKPDLTAALRDVRQQ; SEQ ID NO: 38), or vimentin 415-433 (LPNFSSLNLRETNLDSLPL; SEQ ID NO: 39). In some examples, the vimentin antigen is citrullinated.

In further embodiments, the antigen is α-enolase or an antigenic determinant thereof. A specific non-limiting example of an antigen of use is amino acids 5-21 of α-enolase (KIHAREIFDSRGNPTVE; SEQ ID NO: 40). In some examples, the α-enolase antigen is citrullinated.

In some embodiments, the antigen human cartilage glycoprotein-39 or an antigenic determinant thereof. A specific non-limiting example of an antigen of use is amino acids 259-271 of human cartilage glycoprotein 39 (PTFGRSFTLASSE; SEQ ID NO: 41). In some examples, the cartilage glycoprotein-39 antigen is citrullinated.

In some examples, the antigen is covalently linked to the MHC class II or MHC class I molecule by operably linking a nucleic acid sequence encoding the selected antigen to the 5′ end of the construct encoding the MHC protein such that, in the expressed peptide, the antigenic peptide domain is linked to the amino-terminus of β1 (in the case of β1α1 molecules) or α1 (in the case of α1α2 molecules). One convenient way of obtaining this result is to incorporate a sequence encoding the antigen into the PCR primers used to amplify the MHC coding regions. In some embodiments, a sequence encoding a linker peptide sequence is included between the molecules encoding the antigenic peptide and the MHC polypeptide. As discussed above, the purpose of such linker peptides is to provide flexibility and permit proper conformational folding of the peptides. For linking antigens to the MHC polypeptide, the linker should be sufficiently long to permit the antigen to fit into the peptide groove of the MHC polypeptide. Again, this linker may be conveniently incorporated into the PCR primers. However, it is not necessary that the antigenic peptide be ligated exactly at the 5′ end of the MHC coding region. For example, the antigenic coding region may be inserted within the first few (typically within the first 10) codons of the 5′ end of the MHC coding sequence.

This genetic system for linkage of the antigenic peptide to the MHC molecule is particularly useful where a number of MHC molecules with differing antigenic peptides are to be produced. The described system permits the construction of an expression vector in which a unique restriction site is included at the 5′ end of the MHC coding region (e.g., at the 5′ end of β1 in the case of β1α1-encoding constructs and at the 5′ end of α1 in the case of α1α2-encoding constructs).

In conjunction with such a construct, a library of antigenic peptide-encoding sequences is made, with each antigen-coding region flanked by sites for the selected restriction enzyme. The inclusion of a particular antigen into the MHC molecule is then performed simply by (a) releasing the antigen-coding region with the selected restriction enzyme, (b) cleaving the MHC construct with the same restriction enzyme, and (c) ligating the antigen coding region into the MHC construct. In this manner, a large number of MHC-polypeptide constructs can be made and expressed in a short period of time.

In other examples, the β1α1 and α1α2 molecules are expressed and purified in an empty form (e.g., without attached antigenic peptide), and the antigen is loaded into the molecules using standard methods. Methods for loading of antigenic peptides into MHC molecules are described in, for example, U.S. Pat. No. 5,468,481, herein incorporated by reference. Such methods include simple co-incubation of the purified MHC molecule with a purified preparation of the antigen.

In some examples, the antigen is covalently linked to the MHC molecule by a disulfide bond. In some examples, the disulfide linkage is formed utilizing a naturally occurring cysteine residue in the MHC polypeptide (such as a cysteine residue in the MHC class II β1 domain or a cysteine residue in a MHC class I α1 domain). In some examples, the cysteine residue is in the MHC class II β1 domain or in the MHC class I α1 domain. In particular examples, the disulfide linkage utilizes Cys 17 and/or Cys 79 of a MHC β1α1 polypeptide (for example, SEQ ID NO: 42). In other examples, the disulfide linkage is formed utilizing a non-naturally occurring cysteine residue in the MHC polypeptide, such as a cysteine residue introduced in the MHC polypeptide by mutagenesis. In further examples, the disulfide linkage is formed utilizing a naturally occurring cysteine residue in the peptide antigen. In still further examples, the disulfide linkage is formed utilizing a non-naturally occurring cysteine residue in the peptide antigen, such as a cysteine residue introduced in the peptide antigen by mutagenesis. Exemplary MHC molecules wherein the antigen is covalently linked by a disulfide bond are described in U.S. Provisional Pat. Application No. 61/380,191, filed Sep. 3, 2010, incorporated herein by reference in its entirety.

In one non-limiting example, empty β1α1 molecules (e.g., 1 mg/ml; 40 μM) may be loaded by incubation with a 10-fold molar excess of peptide (e.g., 10 mg/ml; 400 μM) at room temperature, for 24 hours or more. Thereafter, excess unbound peptide may be removed by dialysis against PBS at 4° C. for 24 hours. As is known in the art, peptide binding to β1α1 can be detected and/or quantified by silica gel thin layer chromatography (TLC) using radiolabeled peptide or by gel electrophoresis. Based on such quantification, the loading may be altered (e.g., by changing the molar excess of peptide or the time of incubation) to obtain the desired result.

### V. METHODS OF TREATMENT

Methods are provided herein for treating or inhibiting an autoimmune disease. Exemplary autoimmune diseases, and exemplary antigens of use in these autoimmune diseases, are listed in Table 2

TABLE 2
Exemplary autoimmune disorders and antigens

| Human Disease | Animal Model | Antigen of Use |
|---|---|---|
| Multiple Sclerosis | Experimental | Myelin basic protein (MBP), |
| | autoimmune encephalitis | proteolipid protein (PLP) and |
| | (EAE) mouse model and | myelin oligodendrocyte |
| | Lewis rat | glycoprotein (MOG) |
| Diabetes | NOD mice | Insulin, glutamate decarboxylase |
| Arthritis and related | Chicken, Mice and Rats | Type II collagen, fibrinogen-α, |
| MCTD (mixed | | vimentin, α-enolase, cartilage |
| connective tissue | | glycoprotein-39 |
| disease) | | |
| Hashimoto's Thyroiditis, | Mice, Lewis Rats, and | Thyroglobulin, Thyrodoxin |
| Grave's Disease | OS chickens | |
| Uveitis | Mice | S-antigen, interphotoreceptor |
| | | retinoid binding protein (IRBP) |
| Inflammatory Bowel | MDr1a Knockout Mice | Ach (acetylcholine) Receptor |
| Disease | | |
| Polyarteritis | Mice | HepB Antigen |
| Myasthenia Gravis | Mice | |
| Transplantation rejection | Mice | Insulin, glutamate decarboxylase |
| | Islet cell transplantation | |
| Celiac Disease | Mice expressing a | Cyclooxegenase-2 inhibitor, |
| | transgenic T-cell receptor | dietary hen egg white lysozyme |
| | that recognizes hen egg- | |
| | white lysozyme peptide | |
| | 46-61 | |
| Neuritis | Experimental | Pertussis toxin |
| | autoimmune neuritis | |
| | (EAN) in Lewis rats | |
| Polymyositis | Guinea Pigs, Mice | Myosin B of rabbit shredded |
| | | muscle, Ross River virus (RRV) |
| Sjogren's syndrome | NOD mice, MRL/lpr | |
| | mice | |
| Crohn's disease | SAMP1/Yit mice | |
| Ulcerative colitis | Galphai2(−/−) mice | |
| Glomerulonephritis | Rats | Anti-Gbm serum |
| Autoimmune thyroid | Mice | recombinant murine TPO |
| disease | | (rmTPO) ectodomain |
| Addison's disease | Mice | syngeneic adrenal extract mixed |
| | | with O3 lipopolysaccharide (KO3 LPS) |
| Autoimmune | Experimental | Retinal extract |
| uveoretinitis | Autoimmune | |
| | Uveoretinitis (EAU) | |
| | Lewis rats | |
| Autoimmune pancreatitis | MRL/Mp−+/+(MRL/+) | Polyinosinic:polycytidylic acid |
| | mice | (poly I:C) |
| Primary biliary cirrhosis | C57/BL mice | Lipopolysaccharide (LPS) |
| | | derived from Salmonella |
| | | minnesota Re59 |
| Autoimmune Gastritis | C3H/He mice; | gastric H/K-ATPase, lymphoid |
| (Pernicious anemia) | BALB/c mice | irradiation |
| Hemolytic anemia | CD47-deficient nonobese | |
| | diabetic (NOD) | |

In specific embodiments, methods are provided for treating multiple sclerosis, rheumatoid arthritis, systemic lupus erythematosus, autoimmune hemolytic anemia, pure red cell aplasia, idiopathic thrombocytopenic purpura, Evans syndrome, Wegener's Granulomatosis, pemphigus, type 1 diabetes mellitus, Sjogren's syndrome, and Devic's disease.

In certain embodiments, the administration of an antibody that binds B cells (for example, specifically binds CD20, CD22, CD19, CD40, CD80, or BLyS) and one or more disclosed MHC molecules (RTLs) results in desired clinical effects in the disease or disorder being treated.

In some embodiments, methods are provided herein for the treatment of rheumatoid arthritis (RA). For example, in patients affected by rheumatoid arthritis, administration of one or more antibodies that bind B cells (for example, specifically binds CD20, CD22, CD19, CD40, CD80, or BLyS) and one or more MHC molecules (such as 1, 2, 3, 4, 5, or more of those disclosed herein) improves the patient's condition by a clinically significant amount. In some examples, an improvement of the condition is assessed by the Paulus criteria (improvement in four of: tender and swollen joint counts, morning stiffness, patient assessment of disease activity, physician assessment of disease activity, and erythrocyte sedimentation rate). The level of improvement is set as a percentage improvement of each variable (e.g., a Paulus 20 classification indicates a responder who has shown 20% improvement in four of the parameters). In other examples, an improvement of the condition is assessed by the American College of Rheumatology (ACR) criteria (improvement in the joint counts and improvement in three of: patient assessment, physician assessment, erythrocyte sedimentation rate, pain scale, and functional questionnaire). Improvement is expressed as ACR20, ACR50, or ACR70, indicating an improvement to the 20%, 50%, or 70% level, respectively, in the parameters. Biological measures for improvement in an RA patient after administration include measurement of changes in cytokine levels, measured via protein or RNA levels. Cytokines of interest include, but are not limited to, TNF-α, IL-1, and the interferons.

In RA patients, markers relevant to bone turnover (bone resorption or erosion) can be measured before and after administration of the one or more antibodies that bind B cells (for example, specifically binds CD20, CD22, CD19, CD40, CD80, or BLyS) and the one or more MHC molecules. Relevant markers include, but are not limited to, alkaline phosphatase, osteocalcin, collagen breakdown fragments, hydroxyproline, tartrate-resistant acid phosphatase, and RANK ligand (RANKL). Other readouts relevant to the improvement of RA include measurement of C reactive protein (CRP) levels, serum amyloid A (SAA) levels, erythrocyte sedimentation rate (ESR), rheumatoid factor, CCP (cyclic citrullinated peptide) antibodies and assessment of systemic B cell levels and lymphocyte count via flow cytometry. Specific factors can also be measured from the synovium of RA patients, including assessment of B cell levels in synovium from synovium biopsy, levels of RANKL and other bone factors and cytokines set out above.

When other autoimmune diseases are treated, the outcome of treatment must be measured accordingly. For example, Crohn's disease patients receiving treatment with one or more antibodies that bind B cells (for example, specifically binds CD20, CD22, CD19, CD40, CD80, or BLyS) and one or more MHC molecules achieve an improvement in Crohn's Disease Activity Index (CDAI) in the range of about 50 to about 70 units, wherein remission is at 150 units (Simonis et al, Scand. J Gastroent. 33:283-288, 1998). A score of 150 or 200 is considered normal, while a score of 450 is considered a severe disease score. The use of the methods disclosed herein can result in a reduction in perinuclear anti-neutrophil antibody (pANCA) and anti-Saccharomyces cerevisiae antibody (ASCA) in individuals affected by inflammatory bowel disease.

Methods are provided herein for the treatment of subjects that have multiple sclerosis. In one embodiment the subject has relapsing-remitting multiple sclerosis. However, the methods disclosed herein can also be used for the treatment of subjects with other forms of multiple sclerosis, such as secondary or primary progressive multiple sclerosis. The administration of a therapeutically effective amount of one or more antibodies that bind B cells (for example, specifically binds CD20, CD22, CD19, CD40, CD80, or BLyS) and one or more of the disclosed MHC molecules can achieve an improvement in clinical score on the Kurtzke Expanded Disability status scale (EDSS) (Kurtzke, Neurology 33:1444-52, 1983) of at least 0.5, or a delay in worsening of clinical disease of at least 1.0 on the Kurtzke scale (Rudick et al., Neurology, 49:358-63, 1997). Treatment can also reduce the average rate of increase in the subject's disability score over some period (e.g., 6, 12, 18 or 24 months), e.g., as measured by the EDSS score, by at least about 10% or about 20%, such as by at least about 30%, 40% or 50%. In one embodiment, the reduction in the average rate of increase in the ESS score is at least about 60%, at least about 75%, or at least about 90%, or can even lead to actual improvement in the disability score, compared to control subjects, such as untreated subjects or subjects not receiving the treatment.

In some embodiments, treatment reduces the number of gadolinium enhanced MRI lesions, such as by at least 30%. In one embodiment, the gadolinium enhanced MRI lesions are reduced by at least about 50% or by at least about 70%, such as a reduction of about 80%, about 90%, or by more than 95%, as compared to baseline measurements for the same subjects or to measurement in control subjects (e.g., subjects not receiving the treatment). Similarly, treatment can reduce the average number of MS exacerbations per subject in a given period (e.g., 6, 12, 18 or 24 months) by at least about 25%, such as at least about 40% or at least about 50%. In one embodiment, the number of MS exacerbations is reduced by at least about 80%, such as at least about 90%, as compared to control subjects. The control subjects can be untreated subject or subjects not receiving the treatment (e.g., subjects receiving other agents).

These benefits of treatment with one or more antibodies that bind B cells (for example, specifically binds CD20, CD22, CD19, CD40, CD80, or BLyS) and one or more MHC molecules (for example for RA or MS) can be demonstrated in one or more randomized, placebo-controlled, double-blinded, Phase II or III clinical trials and will be statistically significant (e.g., p<0.05).

The antibody that binds B cells (for example, specifically binds CD20, CD22, CD19, CD40, CD80, or BLyS) can be administered parenterally, e.g., subcutaneously, intramuscularly or intravenously or by means of a needle-free injection device. The compositions for parenteral administration will commonly include a solution of the antibody in a pharmaceutically acceptable carrier. The concentration of antibody in the formulations can vary widely, e.g., from less than about 0.5%, usually at or at least about 1%, to as much as 15 or 20% by weight, or from 1 mg/mL to 100 mg/mL. The concentration is selected primarily based on fluid volumes, viscosities, etc., in accordance with the particular mode of administration selected. Methods for preparing pharmaceutical compositions are known those skilled in the art (see Remington: The Science and Practice of Pharmacy, The University of the Sciences in Philadelphia, Editor, Lippincott, Williams, & Wilkins, Philadelphia, Pa., 21^{st} Edition, 2005).

Antibodies of use in the methods disclosed herein can be frozen or lyophilized for storage and reconstituted in a suitable carrier prior to use. One of skill in the art can readily design appropriate lyophilization and reconstitution techniques.

The one or more antibodies that bind B cells (for example, specifically binds CD20, CD22, CD19, CD40, CD80, or BLyS) and the one or more MHC molecules can be administered for therapeutic treatments of a subject with an autoimmune disease, such as, but not limited to rheumatoid arthritis or multiple sclerosis. Thus, a therapeutically effective amount of a composition is administered to a subject already suffering from the autoimmune disease, such as RA or MS, in an amount sufficient to improve a sign or a symptom of the disorder. Generally a suitable dose of an antibody that binds B cells (for example, specifically binds CD20, CD22, CD19, CD40, CD80, or BLyS) is about 0.5 mg/kg to about 3 mg/kg, such as a dose of about 1 mg/kg, about 1.5 mg/kg, about 2 mg/kg, or about 2.5 mg/kg administered intravenously or subcutaneously. Unit dosage forms are also possible, for example 50 mg, 100 mg, 150 mg or 200 mg, or up to 400 mg per dose. However, other higher or lower dosages also could be used, such as from about 0.5 to about 8 mg/kg.

Single or multiple administrations of the antibody that binds B cells (for example, specifically binds CD20, CD22, CD19, CD40, CD80, or BLyS) can be carried out with dose levels and pattern being selected by the treating physician. Generally, multiple doses are administered. In several examples, multiple administrations are utilized, such as administration monthly, bimonthly, every 6 weeks, every other week, weekly or twice per week. The antibody can be administered systemically or locally, such as orally, subcutaneously or intravenously. However, intra-articular injection can also be utilized. Treatment will typically continue for at least a month, more often for two or three months, sometimes for six months or a year, and may even continue indefinitely, e.g., chronically. Repeat courses of treatment are also possible. The antibody that binds B cells (for example, specifically binds CD20, CD22, CD19, CD40, CD80, or BLyS) can be administered in the same composition as the MHC molecule, or in a separate composition. The antibody that binds B cells (for example, specifically binds CD20, CD22, CD19, CD40, CD80, or BLyS) can be administered concurrently with the MHC molecule, but need not be administered concurrently. The combined administration of the MHC molecule and the antibody that binds B cells (for example, specifically binds CD20, CD22, CD19, CD40, CD80, or BLyS) includes administering the antibody either sequentially with the MHC molecule, e.g., the treatment with one agent first and then the second agent, or administering both agents at substantially the same time, e.g., an overlap in performing the administration. With sequential administration a subject is exposed to the agents at different times so long as some amount of the first agent remains in the subject (or has a therapeutic effect) when the other agent is administered. The treatment with both agents at the same time can be in the same dose, e.g., physically mixed, or in separate doses administered at the same time.

Pharmaceutical compositions that include one or more of the MHC polypeptides (RTLs) disclosed herein (such as 2, 3, 4, 5, or more MHC polypeptides) and/or an antibody that binds B cells (for example, specifically binds CD20, CD22, CD19, CD40, CD80, or BLyS), can be formulated with an appropriate solid or liquid carrier, depending upon the particular mode of administration chosen. The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

The dosage form of the pharmaceutical composition will be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, starch, or magnesium stearate. Actual methods of preparing such dosage forms are known, or will be apparent, to those skilled in the art.

In some examples, the pharmaceutical composition may be administered by any means that achieve their intended purpose. Amounts and regimens for the administration of the selected MHC polypeptides and the antibodies that bind B cells (for example, specifically binds CD20, CD22, CD19, CD40, CD80, and/or BLyS) will be determined by the attending clinician.

With regard to the MHC polypeptide, effective doses for therapeutic application will vary depending on the nature and severity of the condition to be treated, the particular MHC polypeptide selected, the age and condition of the patient, and other clinical factors. Typically, the dose range will be from about 0.1 μg/kg body weight to about 100 mg/kg body weight. Other suitable ranges include doses from about 100 μg/kg to 10 mg/kg body weight. The dosing schedule may vary from once a week to daily depending on a number of clinical factors, such as the subject's sensitivity to the protein. Examples of dosing schedules are about 1 mg/kg administered twice a week, three times a week or daily; a dose of about 5 mg/kg twice a week, three times a week or daily; or a dose of about 10 mg/kg twice a week, three times a week or daily.

The pharmaceutical compositions that include one or more of the disclosed MHC molecules can be formulated in unit dosage form, suitable for individual administration of precise dosages. In one specific, non-limiting example, a unit dosage can contain from about 1 ng to about 500 mg of MHC polypeptide (such as about 10 ng to 100 mg or about 10 mg to 100 mg, for example, about 30 mg or about 60 mg). The amount of active compound(s) administered will be dependent on the subject: being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

The MHC molecules and the antibodies that bind B cells (for example, specifically binds CD20, CD22, CD19, CD40, CD80, or BLyS) can be administered to humans or other animals on whose tissues they are effective in various manners such as topically, orally, intravenously, intramuscularly, intraperitoneally, intranasally, intradermally, intrathecally, subcutaneously, via inhalation or via suppository. In one example, the compounds are administered to the subject subcutaneously. In another example, the compounds are administered to the subject intravenously. The particular mode of administration and the dosage regimen will be selected by the attending clinician, taking into account the particulars of the case (e.g., the subject, the disease, the disease state involved, and whether the treatment is prophylactic). Treatment can involve daily or multi-daily doses of compound(s) over a period of a few days to months, or even years.

An additional agent can be used for the treatment of an autoimmune disease, in addition to the MHC molecule and the antibody that binds B cells (for example, specifically binds CD20, CD22, CD19, CD40, CD80, or BLyS). These agents include immunosuppressive agents, which act to suppress or mask the immune system of the individual being treated. Immunosuppressive agents include, for example, non-steroidal anti-inflammatory drugs (NSAIDs), analgesics, glucocorticoids, disease-modifying antirheumatic drugs (DMARDs) for the treatment of arthritis, or biologic response modifiers. Compositions in the DMARD description are also useful in the treatment of many other autoimmune diseases aside from RA.

Exemplary NSAIDs include, but are not limited to, ibuprofen, naproxen, naproxen sodium, Cox-2 inhibitors such as rofecoxib and celecoxib, and sialylates. Exemplary analgesics include, but are not limited to, acetaminophen, oxycodone, tramadol or propoxyphene hydrochloride. Exemplary glucocorticoids include, but are not limited to, cortisone, dexamethasone, hydrocortisone, methylprednisolone, prednisolone, or prednisone. Exemplary biological response modifiers include, but are not limited to, molecules directed against cell surface markers (e.g., CD4, CD5, CTLA4, etc.), cytokine inhibitors, such as the TNF antagonists (e.g. etanercept (ENBREL®), adalimumab (HUMIRA®), and infliximab (REMICADE®)), chemokine inhibitors, and adhesion molecule inhibitors. The biological response modifiers include monoclonal antibodies as well as recombinant forms of molecules. Exemplary DMARDs include, but are not limited to, azathioprine, cyclophosphamide, cyclosporine, methotrexate, penicillamine, leflunomide, sulfasalazine, hydroxychloroquine, gold (oral (auranofin) and intramuscular), and minocycline.

Thus, for example, in the methods disclosed herein, both single additional agents and combinations of additional agents can be used for treatment of an autoimmune disease, such as rheumatoid arthritis. For example, for rheumatoid arthritis, the subject can be treated with DMARDs such as methotrexate, sulfasalazine, or leflunomide; for treatment of lupus with DMARDs, steroids, cyclophosphamide or mycophenolate mofetil; and for treatment of MS with various disease-modifying agents such as interferons (interferon beta-1a (AVONEX® and REBIF®) or interferon beta-1b (BETASERON® or BETAFERON®), glatiramer acetate (COPAXONE®), mitoxantrone, daclizumab (ZENAPAX®) or natalizumab (TYSABRI®).

The following examples are provided to illustrate certain particular features and/or embodiments. These examples should not be construed to limit the disclosure to the particular features or embodiments described.

### EXAMPLES

### Example 1

### Effect of Anti-CD20 Antibody Treatment in RTL-Treated Mice with Collagen-Induced Arthritis

This example describes the effect of treating mice with collagen-induced arthritis (CIA) with RTL and/or anti-CD20 antibody.

### Methods

Animals: DBA/1LacJ mice were obtained from the Jackson Laboratory (Bar Harbor, Me.) and were maintained in micro-isolators at the animal facility of the Portland Veterans Affairs Medical Center. All protocols were carried out in accordance with institutional guidelines.

Collagen: Bovine collagen was obtained from Chondrex (Redmond, Wash.). The collagen peptide 257-274 was synthesized by F-moc chemistry using an automated peptide synthesizer (PE Applied Biosystems, Foster City, Calif.).

Anti-CD20 antibodies: Rituximab (Genentech, South San Francisco, Calif.) and rabbit anti-CD20 mAb that can cross-react with human and mouse CD20 (Epitomics, Inc., Burlingame, Calif.) were utilized in the present study.

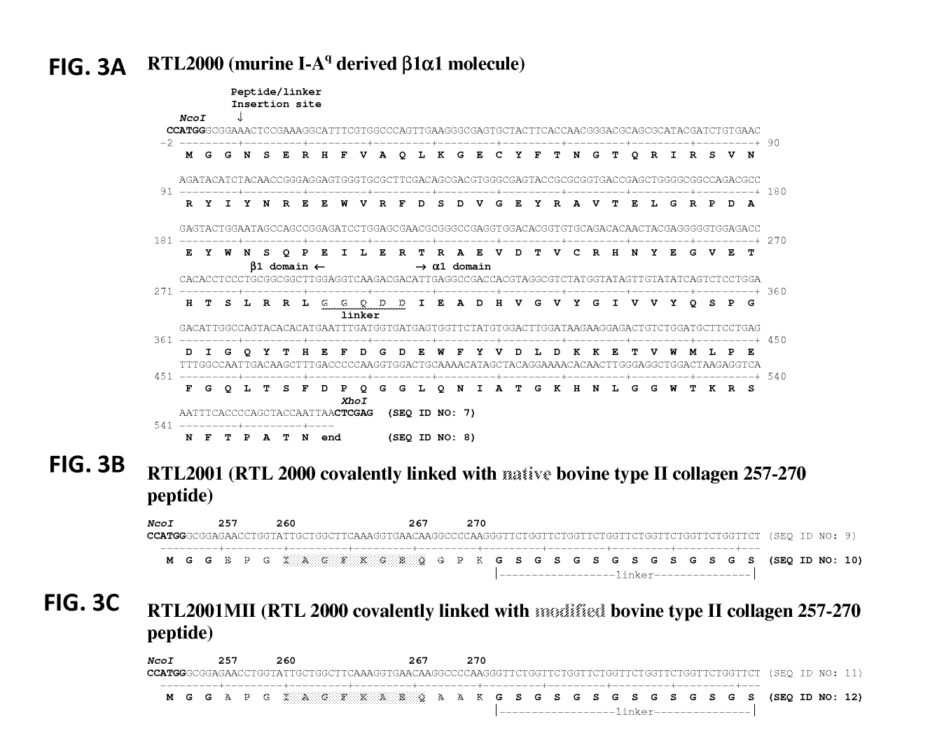

RTL construction, modification and production: General methods for the design, cloning and expression of RTLs have been described previously (e.g., Burrows et al., Prot. Eng. 12:771-778, 1999; Chang et al., J. Biol. Chem. 276:24170-24176, 2001; Huan et al., J. Chem. Technol. Biotechnol. 80:2-12, 2005). In brief, mRNA was isolated from the splenocytes of DBA1/LacJ mice using an Oligotex® Direct mRNA mini kit (Qiagen, Inc., Valencia, Calif.). cDNA of the antigen binding/TCR recognition domain of murine I-A^{q} MHC class II β1 and α1 chains was derived from mRNA using two pairs of PCR primers. The two chains were sequentially linked by a 5 amino acid linker (GGQDD; SEQ ID NO: 43) in a two-step PCR reaction with NcoI and XhoI restriction sites being added to the amino terminus of the β1 chain and to the carboxyl terminus of the α1 chain respectively, to create RTL2000 (FIG. 3A). A linker (GSGSGSGSGSGSGS; SEQ ID NO: 44) and bCII257-270 peptide with or without modification at E257A, G265A, G268A and P269A were covalently linked to the 5′ end of the β1 domain of RTL2000 to form TRL2000 (FIG. 3B) or RTL2001MII (FIG. 3C), respectively. The murine I-A^{q} β1α1 insert was then ligated into pET21d(+) vector and transformed into Nova blue E. coli host (Novagen, Inc., Madison, Wis.) for positive colony selection and sequence verification. RTL2000 and RTL2001MII plasmid constructs were then transformed into E. coli strain BL21 (DE3) expression host (Novagen, Inc., Madison, Wis.). The purification of proteins has been described previously. The final yield of purified protein varied between 30 to 40 mg/L of bacterial culture.

I-A^{q}-derived RTL treatment and arthritis induction: Prior to arthritis induction, male DBA/1LacJ mice between 7-9 weeks age and body weight 20-25 g were randomly divided into two treatment groups. The animals were given a daily intravenous (i.v.) injection for 5 days with either vehicle control (20 mM Tris-Cl buffer) or 100 μl containing 100 μg of RTL2001MII protein. Then, the disease was induced by intradermal (i.d.) injection at the base of tail with 100 μg of bovine collagen II (bCII) protein in Complete Freund's Adjuvant containing 100 μg of Mycobacterium tuberculosis in a 100 μl injection volume. At day 26, the animals were boosted by an injection of 100 μg of bCII protein in Incomplete Freund's Adjuvant in a 100 μl injection volume. Animals were monitored for onset and progression of disease 3-12 weeks post-immunization. The arthritis severity of mice was evaluated with a grading system for each paw according to the following scale: 0=no redness or swelling; 1=slight swelling in ankle or redness in foot; 2=progressive swelling/inflammation and redness from ankle to mid foot; 3=swelling/inflammation of entire foot; 4=swelling and inflammation of entire foot including toes. The Arthritis Score for each mouse was determined by adding the severity scores for each of the four paws.

Anti-CD20 mAb treatment: At day 76 post immunization, four mice from each vehicle control and RTL2001MII pre-treated group were selected based on their similar cumulative disease index (CDI) numbers. These mice were then divided into Rituximab group and anti-mouse CD20 group. In the Rituximab group, the mice were given (i.v.) a single dose of 250 μg Rituximab/mouse, and then 2 days later another single dose of 250 μg Rituximab. In the anti-mouse CD20 mAb group, the mice received (i.v.) a single dose of 250 μg anti-mouse CD20 mAb/mouse. Animals were scored daily as described above for the progression of disease for 3 weeks after treatment.

B cell analysis: The mice were sacrificed at day 98, splenocytes were collected, and the cells were stained with a combination of anti-mouse CD 19 (clone 1D3) and CD45R(B220) (clone RA3-6B2) antibodies, following standard monoclonal antibody staining procedures. Mouse Ig isotypes were used as the control. Fluorescence flow cytometry analyses were performed to determine the percentage of B cell population within splenocytes. Data were acquired with a FACSCalibur™ (Becton-Dickinson, Mountain View, Calif.) and analyzed using FACS express software. All antibodies were purchased from BD Biosciences (San Diego, Calif.).

### Results

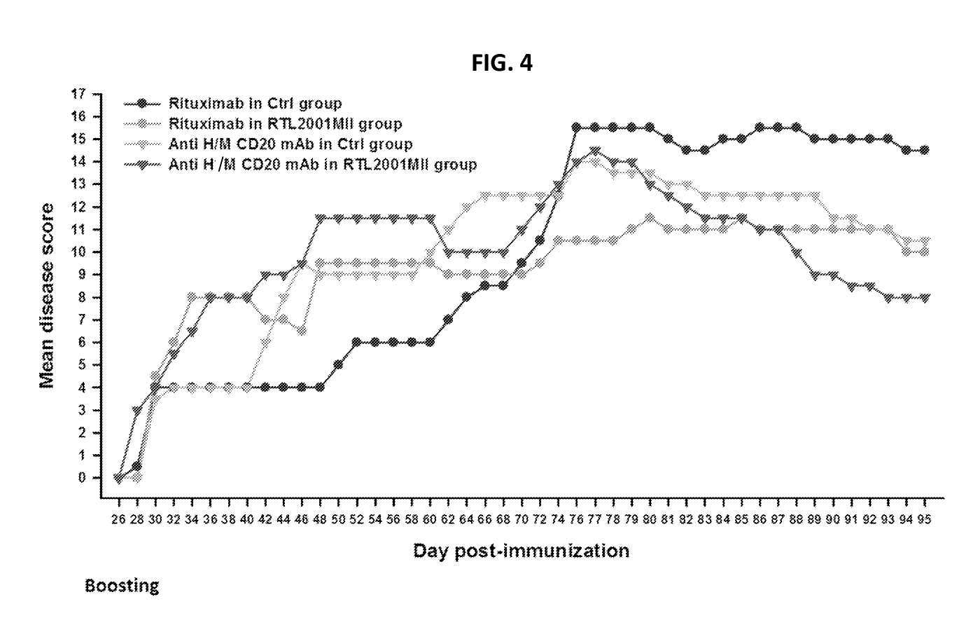

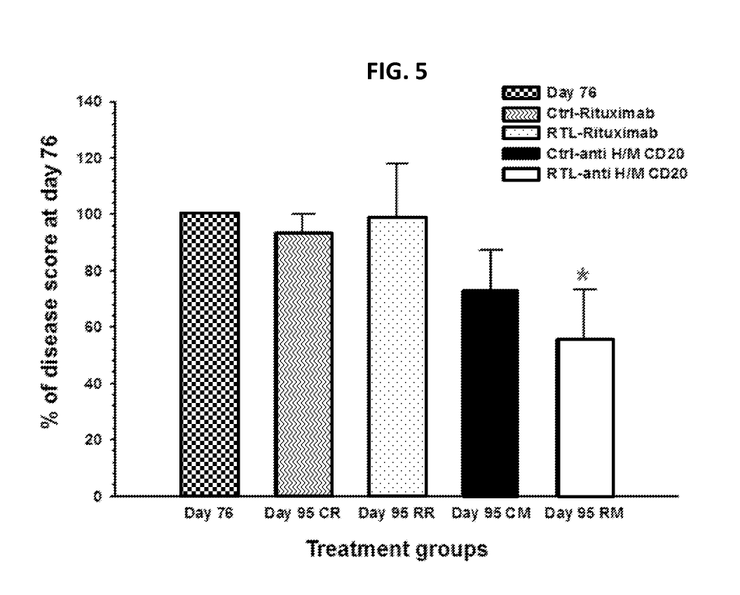

In the current study, a set of monomeric murine I-A^{q}-derived RTLs containing a single chain two-domain MHC class II molecule covalently linked to the immunogenic peptide of bCII257-270 were produced. These novel constructs were used to test the hypothesis that a combination treatment of an RTL and a B cell-targeting mAb can regulate both pathogenic T and B cell activation in a CIA model. As shown in FIG. 4, treatment with a single dose of anti-mouse CD20 mAb can significantly reduce the severity of CIA in the mice that were previous pre-treated with RTL 2001MII. Nineteen days after the mice were treated with anti-mouse CD20 mAb, the average arthritis scores were significantly reduced in the RTL2001MII pre-treated group (44.1%) in comparison to the control group (P<0.01) (FIG. 5). The severity of the disease for the mice from the vehicle control group that were treated with anti-mouse CD20 mAb was also reduced (27.1%) vs. the control but it was not statistically significant. In contrast, Rituximab treatment did not affect the severity of the disease in either the RTL2001MII pre-treated group or in the vehicle control group (FIGS. 4 and 5).

Interestingly, there was no significant change in the percentage of B cells in splenocytes among all treatment groups (Table 3). It has been suggested that anti-CD20 mAb mainly affects B cell population in the peripheral blood but not the spleen. In summary, these results suggest that a combination of RTL that can specifically target antigen-specific T cells and B cell depleting mAbs that can reduce autoantibody production can offer a better treatment approach for RA treatment.

TABLE 3
Effect of anti-CD20 mAb on B cell population

| | | | Score at | | | |
|---|---|---|---|---|---|---|
| | | Day | treat- | Score at | | % CD19+ |
| | Mouse | of | ment | ex vivo | Change | and B220 |
| | ID | onset | (day 76) | (day 98) | in score | population |
| Rituximab/ | 7-0 | ~30 | 15 | 13 | −2 | 74.3 |
| Control | 9-3 | ~30 | 16 | 16 | 0 | 69.2 |
| α-CD20/ | 8-2 | ~30 | 16 | 15 | −1 | 76.0 |
| Control | 9-0 | ~30 | 12 | 4 | −8 | 76.9 |
| Rituximab/ | 5-1 | ~30 | 10 | 7 | −3 | 70.3 |
| Rituximab/ | 5-1 | ~30 | 10 | 7 | −3 | 70.3 |
| RTL2001MII | 6-1 | ~30 | 11 | 10 | −1 | 75.8 |
| α-CD20/ | 4-1 | ~30 | 15 | 10 | −5 | 71.9 |
| RTL2001MII | 4-3 | ~30 | 13 | 7 | −6 | 69.9 |
| Tris alone | 8-1 | ~30 | 2 | 0 | −2 | 84.1 |
| | 9-2 | ~30 | 16 | 11 | −5 | 70.92 |
| RTL2001MII | 4-2 | 76 | 3 | 12 | +9 | 74.4 |
| alone | 5-2 | 74 | 4 | 4 | 0 | 78.7 |
| | 6-0 | 76 | 3 | 11 | +8 | 80.3 |
| Naive | NA | NA | NA | 0 | NA | 76.9 |

NA, not applicable
RTL2001MII alone = Mice pre-treated with 100 μg RTL2001MII daily for 5 days prior to immunization.

### Example 2

### Efficacy of Human-Derived RTL in Reducing Collagen-Induced Arthritis

This example describes the efficacy of a human derived RTL in reducing incidence of collagen-induced arthritis in a mouse model transgenic for human MHC class II DR4.

### Methods

Animals: HLA-DR4 Transgenic (Tg) mice were obtained from Dr. Lars Fugger's Laboratory (University of Oxford, Oxford, UK) and were maintained in micro-isolators at the animal facility of the Portland Veterans Affairs Medical Center. All protocols were carried out in accordance with institutional guidelines.

Collagen: Bovine collagen was obtained from Chondrex (Redmond, Wash.). The collagen peptide 257-274 was synthesized by F-moc chemistry using an automated peptide synthesizer (PE Applied Biosystems, Foster City, Calif.).

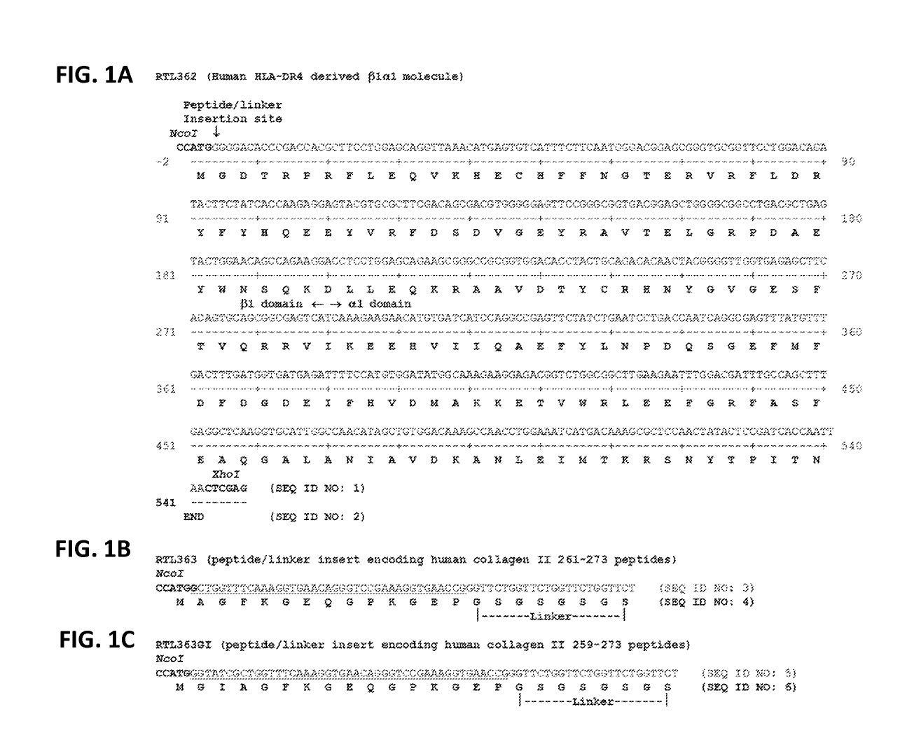

RTL construction, modification and production: General methods for the design, cloning and expression of RTLs have been described previously (e.g., Burrows et al., Prot. Eng. 12:771-778, 1999; Chang et al., J. Biol. Chem. 276:24170-24176, 2001; Huan et al., J. Chem. Technol. Biotechnol. 80:2-12, 2005). In brief, mRNA was isolated from the human genomic DNA using an Oligotex® Direct mRNA mini kit (Qiagen, Inc., Valencia, Calif.). cDNA of the antigen binding/TCR recognition domain of human HLA-DRB1*0401 MHC class II β1 and α1 chains was derived from mRNA using two pairs of PCR primers. The two chains were sequentially linked in a two-step PCR reaction with NcoI and XhoI restriction sites being added to the amino terminus of the β1 chain and to the carboxyl terminus of the α1 chain respectively, to create RTL362 (FIG. 1A). A linker (GSGSGSGS; SEQ ID NO: 45) and bCII261-273 peptide were covalently linked to the 5′ end of the β1 domain of RTL362 to form RTL363 (FIG. 1B). The human HLA-DR4 β1α1 insert was then ligated into pET21d(+) vector and transformed into Nova blue E. coli host (Novagen, Inc., Madison, Wis.) for positive colony selection and sequence verification. RTL362 and RTL363 plasmid constructs were then transformed into E. coli strain BL21 (DE3) expression host (Novagen, Inc., Madison, Wis.). The purification of proteins has been described previously. The final yield of purified protein varied between 20 to 30 mg/L of bacterial culture.

HLA-DR4-derived RTL treatment and arthritis induction: Prior to arthritis induction, DR4 Tg mice between 7-9 weeks age and body weight 20-25 g were randomly divided into three treatment groups (9-10 mice/group). The animals were given a daily i.v. injection for 5 days of 100 μl containing 100 μg of RTL362 protein, 100 μg of RTL363 protein, or vehicle buffer (20 mM Tris-Cl buffer). Then, the disease was induced by intradermal (i.d.) injection at the base of tail with 100 μg of bovine collagen II (bCII) protein in Complete Freund's Adjuvant containing 100 μg of Mycobacterium tuberculosis in a 100 μl injection volume. Animals were monitored for onset and progression of disease 3-12 weeks post-immunization. The arthritis severity of mice was evaluated with a grading system for each paw according to the following scale: 0=no redness or swelling; 1=slight swelling in ankle or redness in foot; 2=progressive swelling/inflammation and redness from ankle to mid foot; 3=swelling/inflammation of entire foot; 4=swelling and inflammation of entire foot including toes. The Arthritis Score for each mouse was determined by adding the severity scores for each of the four paws.

### Results

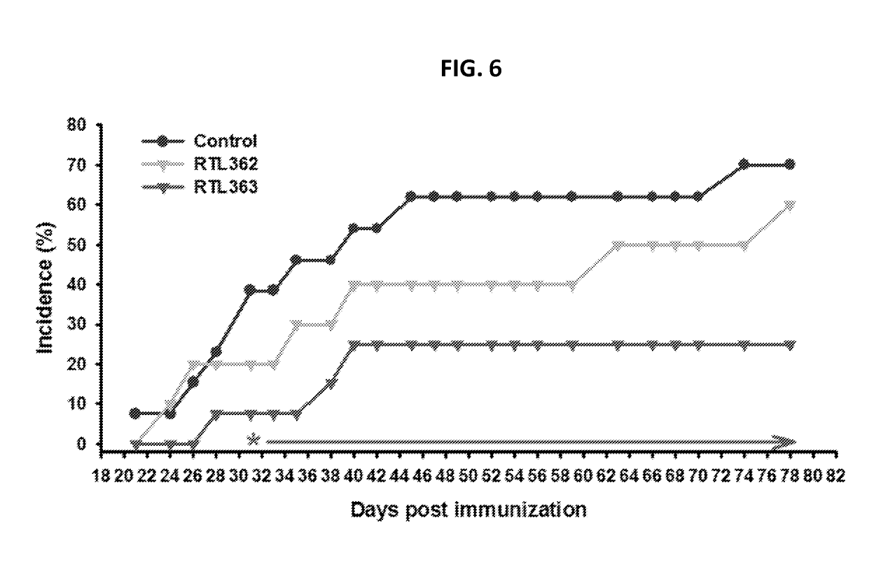

As shown in FIG. 6, the percentage of disease incidence was significantly reduced in the antigen-coupled RTL363 pretreated group (25%) compared to 60% in the “empty” RTL362 pretreated group and 70% in the vehicle control group. The suppression of the disease lasted for more than 78 days post immunization. This result was similar to that observed in DBA1/J mice treated with the RTL2001MII (Example 1).

### Example 3

### Treatment of Rheumatoid Arthritis with RTLs

This example describes exemplary methods for treating rheumatoid arthritis in a subject and exemplary methods for assessing efficacy of RTLs and an antibody that specifically binds CD20 for treating rheumatoid arthritis in a subject. However, one of skill in the art will appreciate that methods that deviate from these specific methods can also be used to treat rheumatoid arthritis in a subject.

Subjects having rheumatoid arthritis are selected. Subjects are treated with an antibody that specifically binds CD20 (such as rituximab), for example, one dose of 1000 mg rituximab i.v. or two doses of 1000 mg rituximab separated by two weeks. Subjects are treated with an RTL i.v. (for example weekly or monthly for 1, 2, 4, 8, 12, 18, 24, or more weeks), for example, an MHC class II β1α1 polypeptide or an MHC class polypeptide covalently linked to an antigen associated with rheumatoid arthritis (such as human collagen II, for example CII261-273) or other RTLs as disclosed herein (such as an MHC class II β1α1 polypeptide covalently linked to a citrullinated fibrinogen-α, vimentin, α-enolase, or cartilage glycoprotein-39 antigen), at doses of 0.1 mg/kg to 10 mg/kg. In some examples, the RTL close is 30 mg or 60 mg.

Subjects are assessed for measures of rheumatoid arthritis (such as utilizing the Paulus criteria or American College of Rheumatology (ACR) criteria, or changes in cytokine levels, for example, TNF-α, IL-1β, IL-6, IL017, and interferon-γ), prior to initiation of therapy, periodically during the period of therapy, and/or at the end of the course of treatment.

The effectiveness of RTL and anti-CD20 antibody therapy to treat or inhibit rheumatoid arthritis in a subject can be demonstrated by an improvement in one or more measures of rheumatoid arthritis (such as a 20%, 50%, or 70% improvement in the Paulus criteria or ACR criteria), a decrease in pro-inflammatory cytokine levels, or a decrease in progression of one or more symptoms, for example, compared to a control, such as an untreated subject or group of subjects with RA, a subject or group of subjects with RA prior to treatment (for example, the same subject or group prior to treatment), or subject or group of subjects with RA treated with placebo (e.g., vehicle only).

### Example 4

### Treatment of Multiple Sclerosis with RTLs

This example describes exemplary methods for treating multiple sclerosis in a subject and exemplary methods for assessing efficacy of RTLs and an antibody that specifically binds CD20 for treating multiple sclerosis in a subject. However, one of skill in the art will appreciate that methods that deviate from these specific methods can also be used to treat multiple sclerosis in a subject.

Subjects having multiple sclerosis are selected. Subjects are treated with an antibody that specifically binds CD20 (such as rituximab), for example, one dose of 1000 mg rituximab i.v. or two doses of 1000 mg rituximab separated by two weeks. Subjects are treated with an RTL i.v. (for example weekly or monthly for 1, 2, 4, 8, 12, 18, 24, or more weeks), for example, an MHC class II β1α1 polypeptide or an MHC class II β1α1 polypeptide covalently linked to a an antigen associated with multiple sclerosis (such as MOG, MBP, or PLP) or other RTLs as disclosed herein, at doses of 0.1 mg/kg to 10 mg/kg. In some examples, the RTL dose is 30 mg or 60 mg.

Subjects are assessed for measures of multiple sclerosis (such as number of exacerbations or disability score), prior to initiation of therapy, periodically during the period of therapy, and/or at the end of the course of treatment.

The effectiveness of RTL and anti-CD20 antibody therapy in subjects with multiple sclerosis can be demonstrated by a reduction in the average number of multiple sclerosis exacerbations per subject in a given period (such as 1, 3, 6, 12, 18 or 24 months) compared to a control, such as an untreated subject or group of subjects, a subject or group of subjects with multiple sclerosis prior to treatment (for example, the same subject or group of subjects prior to treatment), or a subject or group of subjects with multiple sclerosis treated with placebo (e.g., vehicle only). A reduction in the average rate of increase in the subject's disability score over some period (e.g., 1, 3, 6, 12, 18 or 24 months), for example, as measured by the EDSS score, or even an improvement in the disability score, compared to a control, such as an untreated subject or group of subjects, a subject or group of subjects with multiple sclerosis prior to treatment (for example, the same subject or group of subjects prior to treatment), or a subject or group of subjects with multiple sclerosis treated with placebo (e.g., vehicle only) can also demonstrate the effectiveness of RTL and anti-CD20 antibody therapy.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method for treating or inhibiting an autoimmune disease in a subject, comprising:
administering to the subject a therapeutically effective amount of a Major Histocompatibility Complex (MHC) molecule comprising covalently linked first, second and third domains, wherein:
a) the first domain is an MHC class II β1 domain and the second domain is an MHC Class II α1 domain, wherein the amino terminus of the α1 domain is covalently linked to the carboxy terminus of the β1 domain, wherein the MHC molecule does not comprise an MHC class II α2 domain or an MHC Class II β2 domain; or
b) the first domain is an MHC class I α1 domain and the second domain is an MHC class I α2 domain, wherein the amino terminus of the α2 domain is covalently linked to the carboxy terminus of the α1 domain, and wherein the MHC molecule does not comprise an α3 domain; and
the third domain is covalently linked to the first domain, wherein the third domain comprises an antigen associated with the autoimmune disorder; and
administering to the subject a therapeutically effective amount of an antibody that binds B cells, thereby treating or inhibiting the autoimmune disease in the subject.

2. The method of claim 1, wherein the autoimmune disorder is an inflammatory arthropathy or multiple sclerosis.

3. The method of claim 2, wherein the inflammatory arthropathy is rheumatoid arthritis.

4. (canceled)

5. The method of claim 1, wherein the antibody that binds to B cells comprises an antibody that specifically binds to one or more of CD20, CD22, CD19, CD40, CD80, or B-lymphocyte stimulator.

6. The method of claim 5 wherein the antibody that binds to B cells comprises a monoclonal antibody that specifically binds CD20.

7. The method of claim 6, wherein the monoclonal antibody that specifically binds CD20 comprises rituximab.

8. The method of claim 1, wherein the covalent linkage between the first domain and the second domain is provided by a polypeptide linker.

9. The method of claim 1, wherein the covalent linkage between the first domain and the third domain is provided by a polypeptide linker sequence or a disulfide bond.

10. The method of claim 1, wherein the autoimmune disease is rheumatoid arthritis and wherein the antigen comprises collagen, vimentin, fibrinogen-α, α-enolase, cartilage glycoprotein-39, or an antigenic determinant thereof, wherein the antigenic determinant is 8 to 30 amino acids in length and binds the MHC molecule.

11. (canceled)

12. The method of claim 10, wherein the antigen comprises collagen and the antigenic determinant comprises CII 257-270, CII 259-273, CII261-273, or CII 261-274.

13. (canceled)

14. The method of claim 10, wherein the antigen is collagen and the antigenic determinant is glycosylated.

15. (canceled)

16. The method of claim 10, wherein the antigen comprises fibrinogen-α and the antigenic determinant comprises fibrinogen-α 40-59, fibrinogen-α 616-625, fibrinogen-α 79-91 or fibrinogen-α 121-140.

17-18. (canceled)

19. The method of claim 10, wherein the antigen comprises vimentin and the antigenic determinant comprises vimentin 59-79, vimentin 26-44, vimentin 256-275, vimentin 415-433.

20-21. (canceled)

22. The method of claim 10, wherein the antigen comprises α-enolase and the antigenic determinant comprises α-enolase 5-21.

23-24. (canceled)

25. The method of claim 10, wherein the antigen comprises human cartilage glycoprotein-39 and the antigenic determinant comprises human cartilage glycoprotein 39 259-271.

26. (canceled)

27. The method of claim 10, wherein the antigen comprises fibrinogen-α, vimentin, α-enolase, or cartilage glycoprotein-39 and the antigenic determinant is citrullinated.

28. The method of claim 1, wherein the autoimmune disorder is multiple sclerosis, and wherein the antigen comprises a myelin protein or an antigenic determinant thereof, wherein the antigenic determinant is 8 to 30 amino acids in length and binds the MHC molecule.

29. The method of claim 28, wherein the myelin protein comprises myelin oligodendrocyte glycoprotein (MOG), myelin basic protein (MBP), or proteolipid protein (PLP), or an antigenic determinant thereof.

30. The method of claim 29, wherein the antigenic determinant comprises MOG 35-55, MOG 1-25, MOG 94-116, MOG 145-160, MOG 194-208, MBP 10-30; MBP 35-45, MBP 77-91, MBP 85-99, MBP 95-112, MBP 145-164, PLP 139-151, or PLP 95-116.

31-32. (canceled)

33. The method of claim 1, wherein the MHC molecule is an HLA-DR, HLA-DP or HLA-DQ human MHC molecule.

34. (canceled)

35. The method of claim 33, wherein the MHC molecule is modified by substitution of one or more hydrophobic amino acids within a β-sheet platform of the MHC molecules such that the MHC molecule has reduced aggregation in solution compared to aggregation exhibited by an unmodified MHC molecule with a wild-type β-sheet platform.

36. The method of claim 35, wherein the one or more hydrophobic amino acids are V102, I104, A106, F108, and L110, and wherein the one or more hydrophobic amino acids are substituted with a non-hydrophobic amino acid.

37-39. (canceled)

40. The method of claim 1, further comprising treating the subject with an additional immunosuppressive agent.

41. (canceled)


SEQUENCE LISTING (45 sequences)

SEQ ID NO: 1 - DNA, 551 bp, Artificial Sequence
  RTL362 - human HLA-DR4 derived beta1/alpha 1 molecule
ccatgggggacacccgaccacgcttcctggagcaggttaaacatgagtgtcatttcttcaatgggacggagcgggtgcggttcctggacagatacttctatcaccaagaggagtacgtgcgcttcgacagcgacgtgggggagttccgggcggtgacggagctggggcggcctgacgctgagtactggaacagccagaaggacctcctggagcagaagcgggccgcggtggacacctactgcagacacaactacggggttggtgagagcttcacagtgcagcggcgagtcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaactcgag

SEQ ID NO: 2 - protein, 180 aa, Artificial Sequence
  Synthetic Construct
MGDTRPRFLEQVKHECHFFNGTERVRFLDRYFYHQEEYVRFDSDVGEFRAVTELGRPDAEYWNSQKDLLEQKRAAVDTYCRHNYGVGESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 3 - DNA, 68 bp, Artificial Sequence
  RTL363 Peptide/linker insert encoding human collagen II 261-273 peptide
ccatggctggtttcaaaggtgaacagggtccgaaaggtgaaccgggttctggttctggttctggttct

SEQ ID NO: 4 - protein, 22 aa, Artificial Sequence
  Synthetic Construct
MAGFKGEQGPKGEPGSGSGSGS

SEQ ID NO: 5 - DNA, 74 bp, Artificial Sequence
  RTL363GI peptide/linker insert encoding human collagen II 261-273 peptide
ccatgggtatcgctggtttcaaaggtgaacagggtccgaaaggtgaaccgggttctggttctggttctggttct

SEQ ID NO: 6 - protein, 24 aa, Artificial Sequence
  Synthetic Construct
MGIAGFKGEQGPKGEPGSGSGSGS

SEQ ID NO: 7 - DNA, 572 bp, Artificial Sequence
  RTL2000 - mouse I-Aq derived beta1/alpha1 molecule
ccatgggcggaaactccgaaaggcatttcgtggcccagttgaagggcgagtgctacttcaccaacgggacgcagcgcatacgatctgtgaacagatacatctacaaccgggaggagtgggtgcgcttcgacagcgacgtgggcgagtaccgcgcggtgaccgagctggggcggccagacgccgagtactggaatagccagccggagatcctggagcgaacgcgggccgaggtggacacggtgtgcagacacaactacgagggggtggagacccacacctccctgcggcggcttggaggtcaagacgacattgaggccgaccacgtaggcgtctatggtatagttgtatatcagtctcctggagacattggccagtacacacatgaatttgatggtgatgagtggttctatgtggacttggataagaaggagactgtctggatgcttcctgagtttggccaattgacaagctttgacccccaaggtggactgcaaaacatagctacaggaaaacacaacttgggaggctggactaagaggtcaaatttcaccccagctaccaattaactcgag

SEQ ID NO: 8 - protein, 187 aa, Artificial Sequence
  Synthetic Construct
MGGNSERHFVAQLKGECYFTNGTQRIRSVNRYIYNREEWVRFDSDVGEYRAVTELGRPDAEYWNSQPEILERTRAEVDTVCRHNYEGVETHTSLRRLGGQDDIEADHVGVYGIVVYQSPGDIGQYTHEFDGDEWFYVDLDKKETVWMLPEFGQLTSFDPQGGLQNIATGKHNLGGWTKRSNFTPATN

SEQ ID NO: 9 - DNA, 95 bp, Artificial Sequence
  RTL2001 - peptide/linker encoding native bovine type II collagen 257-270 peptide
ccatgggcggagaacctggtattgctggcttcaaaggtgaacaaggccccaagggttctggttctggttctggttctggttctggttctggttct

SEQ ID NO: 10 - protein, 31 aa, Artificial Sequence
  Synthetic Construct
MGGEPGIAGFKGEQGPKGSGSGSGSGSGSGS

SEQ ID NO: 11 - DNA, 95 bp, Artificial Sequence; identical to SEQ ID NO: 9
  RTL2001MII - peptide/linker encoding modified bovine type II collagen 257-270 peptide

SEQ ID NO: 12 - protein, 31 aa, Artificial Sequence; identical to SEQ ID NO: 10
  Synthetic Construct

SEQ ID NO: 13 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 14 - protein, 180 aa, Artificial Sequence
  Human MHC class II beta1/alpha1 molecule
MGDTRPRFLWQPKRECHFFNGTERVRFLDRYFYNQEESVRFDSDVGEFRAVTELGRPDAEYWNSQKDILEQARAAVDTYCRHNYGVVESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 15 - protein, 21 aa, Artificial Sequence
  MOG 35-55 peptide
MEVGWYRPPFSRVVHLYRNGK

SEQ ID NO: 16 - protein, 25 aa, Artificial Sequence
  MOG 1-25 peptide
GQFRVIGPRHPIRALVGDEVELPCR

SEQ ID NO: 17 - protein, 23 aa, Artificial Sequence
  MOG 95-116 peptide
GGFTCFFRDHSYQEEAAMELKVE

SEQ ID NO: 18 - protein, 16 aa, Artificial Sequence
  MOG 145-160 peptide
VFLCLQYRLRGKLRAE

SEQ ID NO: 19 - protein, 15 aa, Artificial Sequence
  MOG 194-208 peptide
LVALIICYNWLHRRL

SEQ ID NO: 20 - protein, 21 aa, Artificial Sequence
  MBP 10-30 peptide
RHGSKYLATASTMDHARHGFL

SEQ ID NO: 21 - protein, 11 aa, Artificial Sequence
  MBP 35-45 peptide
DTGILDSIGRF

SEQ ID NO: 22 - protein, 14 aa, Artificial Sequence
  MBP 77-91 peptide
SHGRTQDENPVVHF

SEQ ID NO: 23 - protein, 15 aa, Artificial Sequence
  MBP 85-99 peptide
ENPVVHFFKNIVTPR

SEQ ID NO: 24 - protein, 18 aa, Artificial Sequence
  MBP 95-112 peptide
IVTPRTPPPSQGKGRGLS

SEQ ID NO: 25 - protein, 20 aa, Artificial Sequence
  MBP 145-164 peptide
VDAQGTLSKIFKLGGRDSRS

SEQ ID NO: 26 - protein, 16 aa, Artificial Sequence
  PLP 139-151 peptide
CHCLGKWLGHPDKFVG

SEQ ID NO: 27 - protein, 22 aa, Artificial Sequence
  PLP 95-116 peptide
GAVRQIFGDYKTTICGKGLSAT

SEQ ID NO: 28 - protein, 14 aa, Artificial Sequence
  collagen II 261-274 peptide
AGFKGEQGPKGEPG

SEQ ID NO: 29 - protein, 15 aa, Artificial Sequence
  Collagen II 259-273 peptide
GIAGFKGEQGPKGEP

SEQ ID NO: 30 - protein, 14 aa, Artificial Sequence
  Collagen II 257-270 peptide
EPGIAGFKGEQGPK

SEQ ID NO: 31 - protein, 14 aa, Artificial Sequence
  Modified collagen II 257-270 peptide
APGIAGFKAEQAAK

SEQ ID NO: 32 - protein, 20 aa, Artificial Sequence
  Fibrinogen-alpha 40-59 peptide
VERHQSACKDSDWPFCSDED

SEQ ID NO: 33 - protein, 20 aa, Artificial Sequence
  Fibrinogen-alpha 616-625 peptide
THSTKRGHAKSRPVRGIHTS

SEQ ID NO: 34 - protein, 13 aa, Artificial Sequence
  Fibrinogen-alpha 79-91 peptide
QDFTNRINKLKNS

SEQ ID NO: 35 - protein, 20 aa, Artificial Sequence
  Fibrinogen-alpha 121-140 peptide
NNRDNTYNRVSEDLRSRIEV

SEQ ID NO: 36 - protein, 21 aa, Artificial Sequence
  Vimentin 59-79 peptide
GVYATRSSAVRLRSSVPGVRL

SEQ ID NO: 37 - protein, 19 aa, Artificial Sequence
  Vimentin 26-44 peptide
SSRSYVTTSTRTYSLGSAL

SEQ ID NO: 38 - protein, 20 aa, Artificial Sequence
  Vimentin 256-275 peptide
IDVDVSKPDLTAALRDVRQQ

SEQ ID NO: 39 - protein, 19 aa, Artificial Sequence
  Vimentin 415-433 peptide
LPNFSSLNLRETNLDSLPL

SEQ ID NO: 40 - protein, 17 aa, Artificial Sequence
  Alpha-enolase 5-21 peptide
KIHAREIFDSRGNPTVE

SEQ ID NO: 41 - protein, 13 aa, Artificial Sequence
  Human cartilage glycoprotein-39 259-271 peptide
PTFGRSFTLASSE

SEQ ID NO: 42 - protein, 185 aa, Artificial Sequence
  Mouse MHC class II beta1/alpha1 molecule
MGGDSERHFVHQFKGECYFTNGTQRIRLVTRYIYNREEYLRFDSDVGEYRAVTELGRHSAEYYNKQYLERTRAELDTACRHNYEETEVPTSLRRLGGEDDIEADHVGFYGTTVYQSPGDIGQYTHEFDGDELFYVDLDKKKTVWRLPEFGQLILFEPQGGLQNIAAEKHNLGILTKRSNFTPATN

SEQ ID NO: 43 - protein, 5 aa, Artificial Sequence
  Linker peptide
GGQDD

SEQ ID NO: 44 - protein, 14 aa, Artificial Sequence
  Linker peptide
GSGSGSGSGSGSGS

SEQ ID NO: 45 - protein, 8 aa, Artificial Sequence
  Linker peptide
GSGSGSGS